(19) United States
(12) Patent Application Publication
(10) Pub. No.: US 2024/0295511 A1
(43) Pub. Date: Sep. 5, 2024

(54) Non-Destructive Inspection Device

(71) Applicant: RIKEN, Wako-shi, Saitama (JP)

(72) Inventors: Yasuo WAKABAYASHI, Wako-shi (JP); Yoshie OTAKE, Wako-shi (JP); Massato TAKAMURA, Wako-shi (JP); Yujiro IKEDA, Wako-shi (JP)

(73) Assignee: RIKEN, Wako-shi, Saitama (JP)

(21) Appl. No.: 18/648,861

(22) Filed: Apr. 29, 2024

(30) Foreign Application Priority Data
Mar. 3, 2023 (JP) 2023-032620

(51) Int. Cl. G01N 23/222; G01N 33/38

(52) U.S. Cl. CPC G01N 23/222; G01N 33/383; G01N 2223/0745; G01N 2223/1013; G01N 2223/106

(57) ABSTRACT

A non-destructive inspection device includes a neutron generation portion, a neutron shield portion, a gamma ray detector, and a gamma ray shield portion. The neutron generation portion emits neutrons spontaneously, or emits neutrons by DD nuclear fusion reaction or DT nuclear fusion reaction. The neutron shield portion is covers the neutron generation portion from at least an area around the neutron generation portion and thereby shields the neutrons at the area, and allows the neutrons to be emitted to a front side of the neutron generation portion. The gamma ray detector detects gamma rays generated in an inspection object on a front side of the neutron generation portion. The gamma rays are generated by the neutrons incident on the inspection object. The neutron shield portion, the gamma ray shield portion, and the gamma ray detector are arranged in this order in alignment with each other in a lateral direction.

### CROSS REFERENCE TO RELATED APPLICATIONS

This application is based on and claims priority under 35 U.S.C. § 119 to Japanese Patent Application No. 2023-032620, filed on Mar. 3, 2023, the entire content of which is incorporated herein by reference.

### BACKGROUND OF THE INVENTION

### 1. Field of the Invention

The present invention relates to a technique of inspecting soundness or the like of an inspection object (e.g., the presence or absence or concentration of a target component in the inspection object) on the basis of gamma rays generated in the inspection object by neutrons incident on the inspection object.

### 2. Description of the Related Art

Damage due to chlorine (chloride ions) is one of factors causing deterioration of infrastructures such as a road and a bridge. The damage due to chlorine is caused by chlorine contained in a sea breeze from a coast, or chlorine contained in an antifreezing agent applied in a cold area or a mountain area. For example, such chlorine infiltrates into a concrete structure constituting an infrastructure. Then, when a concentration of chloride ions around a reinforcing steel bar in the concrete structure exceeds a limit value (a value in a range of 1.2 kg/m³ to 2.5 kg/m³), corrosion of the reinforcing steel bar occurs and progresses, causing the concrete structure to be deteriorated.

For that reason, it is important to inspect a chlorine concentration in a concrete structure and to repair the concrete structure before the corrosion occurs. Such repair can lead to cost reduction or an increase in a service life of the concrete structure (e.g., the above-mentioned bridge).

Patent Literatures 1 and 2 disclose non-destructive inspection devices that can non-destructively inspect presence or absence and a concentration of a target component such as chlorine in a concrete structure. The non-destructive inspection devices of Patent Literatures 1 and 2 can implement the following inspection. Neutrons are made incident on the inspection object. As a result, gamma rays derived from the target component are generated in the inspection object by the neutrons. The gamma rays are detected. Based on the detection result, a depth at which the target component (e.g., chlorine) exists can be determined, and a concentration of the target component at the depth can be acquired.

### CITATION LIST

### Patent Literatures

[Patent Literature 1] International Publication No. 2019/198260
[Patent Literature 2] Japanese Patent Application Laid-Open Publication No. 2021-179345

### SUMMARY OF THE INVENTION

When the non-destructive inspection device implements inspection on the infrastructure as described above, the non-destructive inspection device needs to be transported to the site. Further, the non-destructive inspection device needs to be arranged at each of inspection spots in the site, in order to inspect a large number of the spots in the infrastructure in the site. For the purpose of facilitating such transportation and arrangement of the non-destructive inspection device, a non-destructive inspection device having reduced size and weight is desired.

In view of it, an object of the present invention is to enable a target component in an inspection object to be detected with a non-destructive inspection device whose size and weight are reduced to facilitate transportation and arrangement of the non-destructive inspection device.

In order to achieve the above-described object, a non-destructive inspection device according to the present invention may include a neutron generation portion, a neutron shield portion, a gamma ray detector, and a gamma ray shield portion. The neutron generation portion is configured to spontaneously generate and emit neutrons or to generate and emit neutrons by DD nuclear fusion reaction or DT nuclear fusion reaction. The neutron shield portion is configured to cover the neutron generation portion from at least an area around the neutron generation portion and thereby shield the neutrons at the area around the neutron generation portion, and allow the neutrons to be emitted to a front side of the neutron generation portion. The gamma ray detector is configured to detect gamma rays generated in an inspection object on a front side of the neutron generation portion, and output a detection signal concerning the detection. The gamma rays are generated as a result of the neutrons incident on the inspection object. The neutron shield portion, the gamma ray shield portion, and the gamma ray detector are arranged in this order in alignment with each other in a lateral direction in relation to a forward direction. The forward direction is a direction from a rear side of the neutron generation portion to a front side of the neutron generation portion.

### Advantageous Effect of the Invention

According to the present invention, a target component in an inspection object can be detected with a non-destructive inspection device whose size and weight are reduced to such an extent that the transportation and the disposition thereof are facilitated.

### BRIEF DESCRIPTION OF THE DRAWINGS

FIG. 1A illustrates a configuration example of a non-destructive inspection device according to an embodiment of the present invention.

FIG. 1B is a plan view in the arrows 1B-1B in FIG. 1A.

FIG. 1C is a horizontal sectional view taken along line 1C-1C in FIG. 1A.

FIG. 1D is an illustration of a shape of a gamma ray shield portion.

FIG. 2 is a more detailed configuration diagram of the non-destructive inspection device.

FIG. 3A and FIG. 3B illustrate dimensions of respective parts in a non-destructive inspection device of an implementation example.

FIG. 4 illustrates an energy spectrum of gamma rays acquired by the non-destructive inspection device of the implementation example.

FIG. 5 illustrates a configuration example of a non-destructive inspection device according to a modified example 1.

FIG. 6A and FIG. 6B illustrate a configuration example 1 of a non-destructive inspection device according to a modified example 2.

FIG. 7A to FIG. 7C illustrate a configuration example 2 of the non-destructive inspection device according to the modified example 2.

FIG. 8A to FIG. 8C illustrate a configuration example 3 of the non-destructive inspection device according to the modified example 2.

FIG. 9A to FIG. 9C illustrate a configuration example 4 of the non-destructive inspection device according to the modified example 2.

FIG. 10 illustrates a configuration example of a non-destructive inspection device according to a modified example 3.

FIG. 11A to FIG. 11C illustrate arrangement examples of a neutron source in a non-destructive inspection device according to a modified example 4.

FIG. 12A illustrates a configuration example of a non-destructive inspection device according to a modified example 5.

FIG. 12B is a horizontal sectional view taken along line 12B-12B in FIG. 12A.

FIG. 12C illustrates another configuration example of the non-destructive inspection device according to the modified example 5.

FIG. 12D illustrates another configuration example of the non-destructive inspection device according to the modified example 5.

### DESCRIPTION OF THE PREFERRED EMBODIMENTS

An embodiment of the present invention is described with reference to the drawings. The same reference signs are attached to the parts that are common in the respective drawings, and overlapping description is omitted.

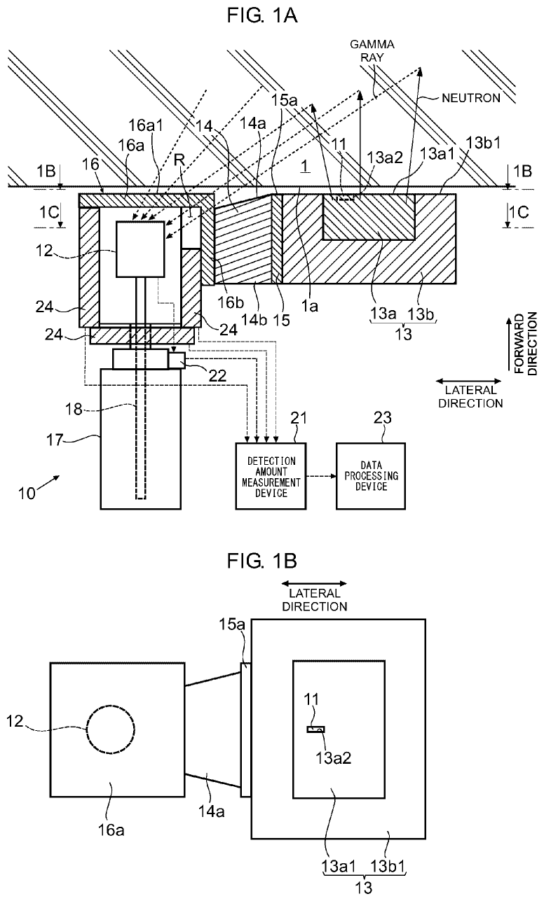

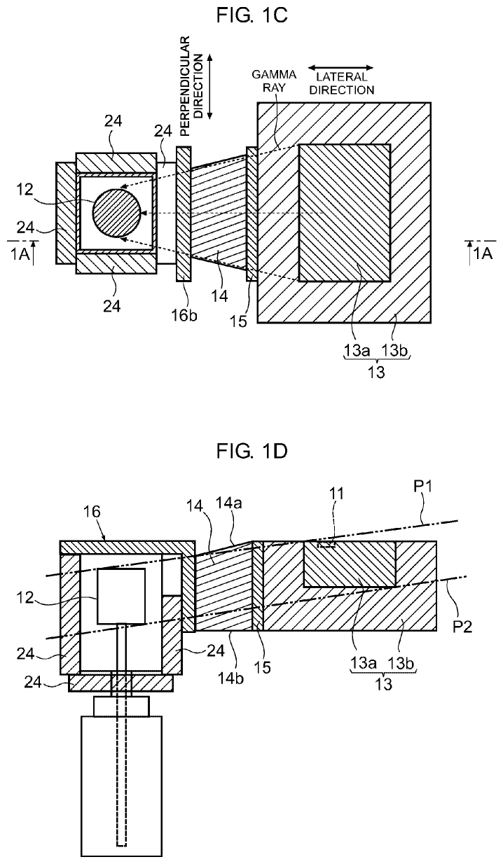

FIG. 1A illustrates a configuration example of a non-destructive inspection device 10 according to the embodiment of the present invention. FIG. 1B is a plan view in the arrows 1B-1B in FIG. 1A. FIG. 1C is a horizontal sectional view taken along the line 1C-1C in FIG. 1A. FIG. 1A is a sectional view taken along line 1A-1A in FIG. 1C.

The non-destructive inspection device 10 is used, at the time of inspection, for causing neutrons (neutron ray) to be incident on a surface 1a of an inspection object 1, detecting gamma rays generated in the inspection object 1 by the neutrons, generating detection signals concerning the detection of the gamma rays, and acquiring detection data, based on the detection signals. The detection data may be an energy spectrum of the gamma rays, as described below. In the present description, the inspection means that the non-destructive inspection device 10 causes neutrons to be incident on the surface 1a of the inspection object 1, and detects the gamma rays generated in the inspection object 1 by the neutrons.

Whether a target component exists in the inspection object 1 can be determined based on the acquired detection data. A concentration of the target component at each depth or a specific depth in the inspection object 1 can be determined based on the acquired detection data. The target component is a component reacting with the neutrons incident on the inspection object 1 and thereby emitting gamma rays having specific energy peculiar to the target component.

The inspection object 1 may be a concrete structure (e.g., a concrete structure constituting a bridge) including reinforcing bars inside, and the target component may be chlorine (or chloride ions). When the target component is chlorine, the chlorine may be a stably existing isotope ³⁵Cl of chlorine Cl, for example. The inspection object 1 and the target component are not limited to a combination of the concrete structure and the chlorine. In other words, the inspection object 1 is not limited to the concrete structure. The target component is also not limited to chlorine Cl, and may be calcium (⁴⁰Ca as a majority), silicon (²⁸Si as a majority), or hydrogen (¹H), for example.

### (Constituents of Non-Destructive Inspection Device)

The non-destructive inspection device 10 includes a neutron source 11, a gamma ray detector 12, a neutron shield portion 13, a gamma ray shield portion 14, and first and second neutron absorption portions 15 and 16.

The neutron source 11 is a radioisotope (RI) neutron source that spontaneously generates and emits neutrons (neutron ray). The RI neutron source may be a spontaneous-fission neutron source. The RI neutron source may be californium (²⁵²Cf). However, according to the present invention, the RI neutron source is not limited to this, and may be an Am—Be neutron source, for example. The neutron source 11 constitutes a neutron generation portion in the present invention.

The gamma ray detector 12 detects the gamma rays generated in the inspection object 1 by the neutrons from the neutron source 11, and thus outputs detection signals concerning the detection. In other words, the gamma ray detector 12 detects the gamma rays incident on the gamma ray detector 12, and outputs a detection signal each time the gamma ray is detected. The detection signal indicates that the gamma ray is incident on the gamma ray detector 12. The detection signal includes information indicating energy of the gamma ray. For example, the detection signal may be a waveform signal that has a pulse height depending on the energy of the gamma ray.

The gamma ray detector 12 may be a germanium semiconductor detector configured by a germanium semiconductor. In this case, the gamma ray detector 12 may be used in a state of being cooled by a cryogenic liquid (e.g., liquid nitrogen). In other words, as illustrated in FIG. 1A, the gamma ray detector 12 may be used in a state of being cooled by the cryogenic liquid via a cold finger (copper rod) 18 immersed in the cryogenic liquid in a dewar 17. Instead of the configuration in which the germanium semiconductor detector 12 is cooled by the cryogenic liquid in this manner, an electric cooler that electrically cools the germanium semiconductor detector 12 may be provided. In this case, the electric cooler may be supported by the below-described support 31.

The gamma ray detector 12 may be configured by a semiconductor material (e.g., Si, CdTe, or CdZnTe) other than germanium, or may be configured by a scintillation detector (e.g., NaI (Tl), BGO, or CsI).

The neutron shield portion 13 covers the neutron source 11 from an area around and a rear side of the neutron source 11, and thereby shields neutrons at the area around and the rear side of the neutron source 11. Meanwhile, the neutron shield portion 13 allows neutrons to be emitted to a front side of the neutron source 11. In this state, the neutron source 11 may be attached to the neutron shield portion 13 (the below-described deceleration portion 13a). A direction from a rear side to a front side of the neutron source 11 is defined as a forward direction. The gamma ray detector 12 and the neutron shield portion 13 are arranged in alignment with each other in a lateral direction (the left-right direction in FIG. 1A) in relation to the forward direction.

In the present description, the forward direction may be a direction (a direction perpendicular to the neutron emission surface 13a1) in which a neutron emission surface 13a1 of the below-described deceleration portion 13a faces, and the lateral direction may be a direction perpendicular to the forward direction. In the present description, concerning the non-destructive inspection device 10, the matter that a first constituent (e.g., the neutron shield portion 13) covers a second constituent (e.g., the neutron source 11) from an area around the second constituent may mean that the first constituent covers the second constituent in each direction (e.g., all the directions perpendicular to the forward direction) perpendicular to the forward direction.

The neutron shield portion 13 may be configured to include the deceleration portion 13a and a reflection portion 13b.

The deceleration portion 13a is formed of a material (a neutron deceleration material that reduces energy of neutrons) that decelerates neutrons. The neutron deceleration material may be a material that has a tendency of not absorbing neutrons. The neutron deceleration material is polyethylene for example, but may be any of other materials. The neutron deceleration material may be a material that has a higher tendency of not absorbing neutrons than the material of the below-described first neutron absorption portion 15.

The deceleration portion 13a covers the neutron source 11 from an area around and a rear side of the neutron source 11. The neutron source 11 may be arranged at a front end portion of the deceleration portion 13a in the forward direction. In this case, the neutron source 11 does not need to be covered with the deceleration portion 13a from a front side (from a forward-direction side). In this case, the neutron source 11 may be exposed to the surface 1a of the inspection object in the forward direction at the time of the inspection.

The deceleration portion 13a includes a neutron emission surface 13a1 facing in the forward direction. The neutron emission surface 13a1 emits, to a front side, neutrons that are included in neutrons emitted from the neutron source 11 and that have made one or more round trips in each of which the neutrons advance to the reflection portion 13b from the deceleration portion 13a, are reflected by the reflection portion 13b, and thereby return to the deceleration portion 13a.

More specifically, a recess 13a2 may be formed on the neutron emission surface 13a1, and the neutron source 11 may be arranged in the recess 13a2. Thereby, the deceleration portion 13a covers the neutron source 11 from the area around and the rear side of the neutron source 11. The recess 13a2 may have a shape that matches with a shape of the neutron source 11. The recess 13a2 may be open to a front side. The neutron source 11 may be bonded to the deceleration portion 13a.

When viewed in a direction (i.e., a rearward direction) opposite to the forward direction, the neutron source 11 is arranged at a position shifted from a lateral-direction center of the deceleration portion 13a to a side of the gamma ray detector 12. Thereby, a distance between the neutron source 11 and the gamma ray detector 12 is shortened. However, a position of the neutron source 11 in the lateral direction is not limited to this, and may be, for example, at the lateral-direction center of the deceleration portion 13a. The neutron source 11 and the gamma ray detector 12 may be arranged on the same imaginary plane parallel to both of the forward direction and the lateral direction (the left-right direction in FIG. 1A).

The reflection portion 13b is formed of a neutron reflection material which is a material that reflects neutrons. The neutron reflection material may be a material that has a tendency of not absorbing neutrons. The neutron reflection material is graphite in an implementation example, but may be any of other materials. The neutron reflection material may be a material that has a higher tendency of reflecting neutrons than the material of the deceleration portion 13a. The neutron reflection material may be a material that has a higher tendency of not absorbing neutrons than the material of the below-described first neutron absorption portion 15.

The reflection portion 13b covers the deceleration portion 13a from an area around and a rear side of the deceleration portion 13a.

A front surface (i.e., the neutron emission surface 13a1) included in the deceleration portion 13a and facing in the forward direction and a front surface 13b1 included in the reflection portion 13b and facing in the forward direction may substantially constitute the same imaginary plane (flat surface). In this case, a front end (front surface) of the neutron source 11 in the forward direction may be located on the same imaginary plane. Each of the front surfaces 13a1 and 13b1 may be a flat surface, but is not limited to this, and may be a curved surface.

The gamma ray shield portion 14 is arranged between the neutron shield portion 13 and the gamma ray detector 12 in the lateral direction, and shields gamma rays. The gamma ray shield portion 14 is formed of a material that shields gamma rays. This material may be lead for example, but may be any of other materials.

The gamma ray shield portion 14 may include an inclined surface 14a as a front end surface as illustrated in FIG. 1A. The inclined surface 14a faces in a direction inclined from the forward direction to a side of the gamma ray detector 12. The inclined surface 14a extends in such a way as to approach the gamma ray detector 12 as a position shifts from a front end in the gamma ray shield portion 14 in the forward direction to a side opposite to this front end. In other words, a front portion of the gamma ray shield portion 14 is formed in such a way that a section of the gamma ray shield portion 14 defined as a section cut by a plane perpendicular to the forward direction has an area that gradually decreases as a position shifts to a front side.

The first neutron absorption portion 15 is arranged between the neutron shield portion 13 and the gamma ray shield portion 14 in the lateral direction. The first neutron absorption portion 15 is formed of a material that absorbs neutrons. This material may be boron carbide (B₄C). For example, the first neutron absorption portion 15 may be formed of rubber containing B₄C. However, the material of the first neutron absorption portion 15 may be a material other than B₄C as long as the material absorbs neutrons.

The second neutron absorption portion 16 covers the gamma ray detector 12 from a front side, and also covers the gamma ray detector 12 from a side of the neutron shield portion 13. The second neutron absorption portion 16 includes a front absorption portion 16a and a lateral absorption portion 16b. The front absorption portion 16a extends in the lateral direction in such a way as to cover the gamma ray detector 12 from a front side. The lateral absorption portion 16b extends in the forward direction in such a way as to cover the gamma ray detector 12 from a side of the neutron shield portion 13 in the lateral direction. A lateral end portion of the front absorption portion 16a on a side of the neutron shield portion 13 is connected to a front end portion of the lateral absorption portion 16b. The front absorption portion 16a may be omitted. In this case, the second neutron absorption portion 16 may consist of the lateral absorption portion 16b.

The second neutron absorption portion 16 may be arranged closer to the gamma ray detector 12 than the gamma ray shield portion 14. The second neutron absorption portion 16 (i.e., the front absorption portion 16a and the lateral absorption portion 16b) is formed of a material that absorbs neutrons. In the implementation example, this material is a material that has a tendency of not generating gamma rays by the incident neutrons even though the material absorbs the neutrons. In this case, this material may be lithium fluoride (LiF). However, the material of the second neutron absorption portion 16 may be an appropriate material other than LiF.

A front surface of the non-destructive inspection device 10 may be configured in such a way that the above-described front surfaces (front surfaces 13a1 and 13b1) included in the neutron shield portion 13 and facing in the forward direction, the front end (an end in the forward direction) of the gamma ray shield portion 14, a front surface 15a of the first neutron absorption portion 15 facing in the forward direction, and a front surface 16a1 of the front absorption portion 16a facing in the forward direction are substantially entirely included in the same imaginary plane (flat surface). Thereby, the entire front surface of the non-destructive inspection device 10 is easily made adjacent to the surface 1a1 of the inspection object 1.

According to the present embodiment, at least respective parts of the gamma ray detector 12, the neutron shield portion 13 (the deceleration portion 13a), the gamma ray shield portion 14, the first neutron absorption portion 15, and the lateral absorption portion 16b are arranged in alignment with each other in the lateral direction. In other words, the gamma ray detector 12, the neutron shield portion 13 (the deceleration portion 13a), the gamma ray shield portion 14, the first neutron absorption portion 15, and the lateral absorption portion 16b at least partially overlap with each other in the lateral direction.

In this case, the gamma ray detector 12, the gamma ray shield portion 14, the neutron shield portion 13, the first neutron absorption portion 15, and the lateral absorption portion 16b may be arranged in alignment with each other in the lateral direction in such a way that when viewed in the lateral direction, each of the gamma ray detector 12, the gamma ray shield portion 14, the neutron shield portion 13, the first neutron absorption portion 15, and the lateral absorption portion 16b is entirely or partially within a range of the neutron shield portion 13.

### (Dimension, Shape, and the Like of Gamma Ray Shield Portion)

A dimension, a shape, and arrangement of the gamma ray shield portion 14 may be set in such a way that the entire gamma ray detector 12 is hidden by the gamma ray shield portion 14 when the gamma ray detector 12 is viewed from any viewpoint (position) in the deceleration portion 13a. With reference to FIG. 1D, the following describes positions of the front end surface (inclined surface) 14a and a rear end surface 14b of such a gamma ray shield portion 14. FIG. 1D corresponds to FIG. 1A, and is an illustration for the front end surface (inclined surface) 14a and the rear end surface 14b.

An imaginary plane in contact with both a front portion of the gamma ray detector 12 and a front portion of the deceleration portion 13a is defined as a front reference plane P1 (the two-dot chain line in FIG. 1D). Each position on the inclined surface 14a of the gamma ray shield portion 14 may be included in the reference plane P1 or located on a front side of the reference plane P1. In this case, the inclined surface 14a may be formed to be entirely included in the reference plane P1.

Similarly, an imaginary plane in contact with both a rear portion of the gamma ray detector 12 and a rear portion of the deceleration portion 13a is defined as a rear reference plane P2 (the two-dot chain line in FIG. 1D). Each position on the rear end surface 14b of the gamma ray shield portion 14 may be included in the reference plane P2 or located on a rear side of the reference plane P2. In this case, the rear end surface 14b may be formed to be entirely included in the reference plane P2.

As long as the entire gamma ray detector 12 is hidden by the gamma ray shield portion 14 when the gamma ray detector 12 is viewed from any viewpoint (position) in the deceleration portion 13a, a perpendicular-direction dimension of the gamma ray shield portion 14 may be made smaller than a perpendicular-direction dimension of the deceleration portion 13a, as illustrated in FIG. 1C. Thereby, an amount of the gamma rays entering the gamma ray detector 12 from the inspection object 1 can be increased while gamma rays from the deceleration portion 13a to the gamma ray detector 12 are shielded. In this case, a perpendicular-direction dimension of the gamma ray shield portion 14 may become smaller as a position shifts to a side of the gamma ray detector 12 in the lateral direction, as illustrated in FIG. 1C. Alternatively, a perpendicular-direction dimension of the gamma ray shield portion 14 may be constant. However, a perpendicular-direction dimension of the gamma ray shield portion 14 is not limited to such dimensions, and may be equal to or larger than a perpendicular-direction dimension of the deceleration portion 13a.

When a distance adjustment mechanism 25 that enables adjustment of a lateral-direction distance from the gamma ray detector 12 to the neutron shield portion 13 and the gamma ray shield portion 14 is provided as in the below-described modified example 1, a dimension, a shape, and arrangement of the gamma ray shield portion 14 may be set as described above. This setting may be made in such a way that the entire gamma ray detector 12 is hidden by the gamma ray shield portion 14 when the gamma ray detector 12 is viewed from any viewpoint (position) in the deceleration portion 13a in a state where the lateral-direction distance becomes the maximum in a adjustable range thereof.

### (Dimension of Gamma Ray Detector)

A perpendicular-direction dimension of the gamma ray detector 12 may be smaller than a perpendicular-direction dimension of each of the neutron shield portion 13, the gamma ray shield portion 14, the first neutron absorption portion 15, and the lateral absorption portion 16b. Similarly, a forward-direction dimension of the gamma ray detector 12 may be smaller than a forward-direction dimension of each of the neutron shield portion 13, the gamma ray shield portion 14, the first neutron absorption portion 15, and the lateral absorption portion 16b. In this case, when viewed in the lateral direction, the gamma ray detector 12 may be located entirely or partially within a range of each of the neutron shield portion 13, the gamma ray shield portion 14, the first neutron absorption portion 15, and the lateral absorption portion 16b. In this case, the rear portion of the gamma ray detector 12 may be located on a rear side of the neutron shield portion 13.

### (Mutual Coupling of Respective Portions)

The gamma ray detector 12, the neutron shield portion 13, the gamma ray shield portion 14, and the first and second neutron absorption portions 15 and 16 may be integrated with each other by being coupled to each other directly or via other members.

(Configuration of Processing Detection Signals from Gamma Ray Detector)

For processing the detection signals, the non-destructive inspection device 10 may further include a detection amount measurement device 21 and a data processing device 23 in addition to the above-described configuration.

Each detection signal generated by the gamma ray detector 12 is input to the detection amount measurement device 21. In this case, each detection signal generated and output by the gamma ray detector 12 may be input to the detection amount measurement device 21 via a preamplifier 22. Thereby, each detection signal amplified by the preamplifier 22 is input to the detection amount measurement device 21.

The detection amount measurement device 21 generates detection data indicating a detection amount of gamma rays (the number of times of detecting a gamma ray) at each energy of the gamma ray, based on each detection signal (e.g., a pulse height of each detection signal) from the gamma ray detector 12. The detection data may be an energy spectrum of the gamma rays. The energy spectrum of the gamma rays is data representing the detection amount of the gamma rays for each energy of the gamma ray. The energy spectrum of the gamma rays may be acquired by performing inspection for a predetermined measurement time (e.g., 10 minutes or more). A device (not illustrated) that applies an operation voltage to the gamma ray detector 12 may be integrated with the detection amount measurement device 21 or may be provided separately from the detection amount measurement device 21.

The energy spectrum is input from the detection amount measurement device 21 to the data processing device 23. The data processing device 23 may determine a depth at which the target component (e.g., chlorine) exists in the inspection object 1, based on the energy spectrum. In addition or alternatively, the data processing device 23 may determine a concentration of the target component at the determined depth or at a specific depth. Here, a method in which the data processing device 23 determines a depth of existence of the target component, based on the energy spectrum, and a method in which the data processing device 2 determines a concentration of the target component at the determined depth or at a specific depth, based on the energy spectrum are described in Patent Literature 1 and Patent Literature 2, and thus, the description thereof is omitted.

### (Configuration for Excluding Detection of Compton-Scattered Gamma Rays)

The non-destructive inspection device 10 may further include a sub-detector 24. In addition to the above-described configuration. The sub-detector 24 is provided for acquiring detection data having a reduced background of gamma rays that are Compton-scattered in the gamma ray detector 12.

Here, the following area R (e.g., the area R in FIG. 1A) is defined as an entry area for the gamma rays from the inspection object 1. When viewed from a forward-direction front end portion of the gamma ray detector 12, the area R is located on an obliquely front side that is a side inclined from the forward direction toward the neutron shield portion 13. The sub-detector 24 covers the gamma ray detector 12 from an area that is around the gamma ray detector 12, but that excludes at least the entry area R. In other words, the sub-detector 24 is arranged in such a way as to surround a center axis passing through the gamma ray detector 12 in the forward direction, but in such a way as not to exist in the entry area R. Accordingly, the gamma rays that advance from inside the inspection object 1 through the entry area R to the gamma ray detector 12 enter the gamma ray detector 12 without entering the sub-detector 24.

The sub-detector 24 may be provided also on a directly rear side of the gamma ray detector 12 in the direction opposite to the forward direction. The sub-detector 24 does not need to be provided on a directly front side of the gamma ray detector 12 in the forward direction. However, the sub-detector 24 may be provided also on a directly front side of (in front of) the gamma ray detector 12.

When a gamma ray is incident on the sub-detector 24, the sub-detector 24 inputs an incidence signal to the detection amount measurement device 21. As described above, when a gamma ray is incident on the gamma ray detector 12, the gamma ray detector 12 inputs the above-described detection signal to the detection amount measurement device 21. When the detection signal and the incidence signal are simultaneously input to the detection amount measurement device 21, the detection amount measurement device 21 discards the detection signal, and does not use the discarded detection signal in generating the detection data. Thereby, the detection of the gamma ray that is among the gamma rays incident on the gamma ray detector 12 and that Compton-scattered and thereby incident on the sub-detector 24 outside the gamma ray detector 12 is not used in generating the above-described detection data. Thereby, reliability of identifying a nuclide, based on the detection data is enhanced. The data processing device 23 may perform the processing of the detection amount measurement device 21. In other words, the detection amount measurement device 21 may be incorporated into the data processing device 23.

A mode of the detection amount measurement device 21 may be set to either of the following first and second modes by a person operating an appropriate operation unit (such as a button or a touch panel). The detection amount measurement device 21 set in the first mode does not use a detection signal in generating the detection data when this detection signal and an incidence signal are simultaneously input to the detection amount measurement device 21. The detection amount measurement device 21 set in the second mode uses a detection signal in generating the detection data even when this detection signal and an incidence signal are simultaneously input to the detection amount measurement device 21.

The sub-detector 24 may be a scintillator formed of bismuth germanate (BGO), but is not limited to this as long as the sub-detector 24 can detect incident gamma rays. The sub-detector 24 may be formed of a material (e.g., BGO) that shields gamma rays from the outside. Thereby, the sub-detector 24 also has a function of suppressing background gamma rays (e.g., gamma rays generated in the deceleration portion 13a) from entering the gamma ray detector 12.

### (Weight of Non-Destructive Inspection Device)

A combination of the neutron source 11, the gamma ray detector (germanium semiconductor detector) 12, the neutron shield portion 13, the gamma ray shield portion 14, the first neutron absorption portion 15, and the second neutron absorption portion 16 is defined as a main structure unit. A weight of the main structure unit may be smaller than 20 kg and equal to or larger than 10 kg (or be smaller than 20 kg and equal to or larger than 15 kg), for example, as in the below-described implementation example, but is not limited to this range, and may be, for example, smaller than 100 kg and equal to or larger than 50 kg, smaller than 50 kg and equal to or larger than 30 kg, or smaller than 30 kg and equal to or larger than 20 kg. In this case, the second neutron absorption portion 16 constituting the main structure unit may be a combination of the front absorption portion 16a and the lateral absorption portion 16b, or may consist of only the lateral absorption portion 16b when the front absorption portion 16a is omitted.

A weight of the sub-detector 24 is approximately 20 kg for example, and a weight of the liquid nitrogen dewar 17 is approximately in a range from 7 kg to 8 kg for example. Thus, a total weight of the main structure unit, the sub-detector 24, and the liquid nitrogen dewar 17 can be set smaller than 50 kg for example.

### (Configuration of Supporting Respective Constituents of Non-Destructive Inspection Device)

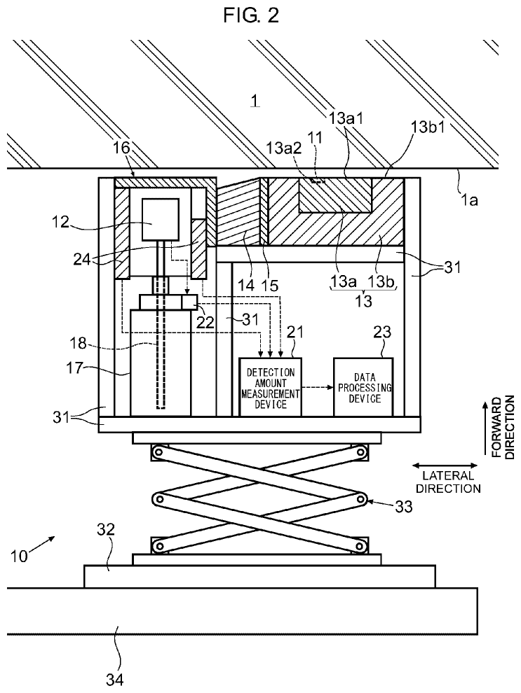

FIG. 2 illustrates the non-destructive inspection device 10 in more detail in FIG. 1A. In the example of FIG. 2, the above-described forward direction is a vertically upward direction, and the surface 1a of the inspection object 1 is a lower surface of the inspection object 1.

As illustrated in FIG. 2, the non-destructive inspection device 10 may further include a support 31 in addition to the above-described configuration. The support 31 supports the neutron shield portion 13, the gamma ray detector 12, the gamma ray shield portion 14, the first and second neutron absorption portions 15 and 16, and the like. The gamma ray detector 12, the neutron shield portion 13, the gamma ray shield portion 14, and the first and second neutron absorption portions 15 and 16 may be integrated with each other by being attached to (e.g., fixed to) the support 31. The detection amount measurement device 21 and the data processing device 23 may be supported on the support 31 as illustrated in FIG. 2.

### (Position Adjustment Mechanism)

As illustrated in FIG. 2, the non-destructive inspection device 10 may further include a base 32 and a position adjustment mechanism 33 in addition to the above-described configuration.

The base 32 supports the support 31 via the position adjustment mechanism 33, and thereby supports the gamma ray detector 12, the neutron shield portion 13, the gamma ray shield portion 14, the first and second neutron absorption portions 15 and 16, and the like. In the example of FIG. 2, the base 32 is installed on a bed 34. The bed 34 is coupled to, for example, a distal end portion of a multi-joint movable boom provided in a vehicle. In this case, the bed 34 may be moved directly below the lower surface 1a of the inspection object 1 (e.g., a bridge) by operation of the movable boom. The bed 34 may be configured to allow a person to ride on the bed 34, in addition to placing the non-destructive inspection device 10 on the bed 34.

The position adjustment mechanism 33 enables a position of the support 31 to be adjusted relative to the base 32 in a direction parallel to the forward direction. Thereby, the position adjustment mechanism 33 enables positions of the gamma ray detector 12, the neutron shield portion 13, the gamma ray shield portion 14, and the first and second neutron absorption portions 15 and 16 to be adjusted relative to the base 32 and be thereby adjusted relative to the surface 1a of the inspection object 1.

When the forward direction is the vertically upward direction, the position adjustment mechanism 33 may be a jack that extends and contracts in the vertical direction as illustrated in FIG. 2. The extension and contraction of the jack may be operated by an operation unit. A person operates the operation unit (not illustrated) to extend and contract the jack by an amount depending on the operation. The jack may be a hydraulic jack or an electric jack, and the operation unit may be a pedal, a lever, a button, or the like. The operation unit may be provided on the jack, or may be provided separately from the jack to operate the jack wirelessly.

The position adjustment mechanism 33 is not limited to the jack, and may have any of other configurations as long as the position adjustment mechanism 33 enables a position of the support 31 to be adjusted relative to the base 32 by moving the support 31 in the direction parallel to the forward direction. For example, the position adjustment mechanism 33 may be constituted by an appropriate linear actuator that moves the support 31 in the direction parallel to the forward direction.

In the present application, the forward direction does not need to be the vertically upward direction, and may be a horizontal direction, an oblique direction inclined from the horizontal direction, or a vertically downward direction.

### Implementation Example

In the non-destructive inspection device 10 of an implementation example, californium (²⁵²Cf) of 3.7 MBq is used as the neutron source 11. In the non-destructive inspection device 10 of the implementation example, the deceleration portion 13a is formed of polyethylene, the reflection portion 13b is formed of graphite, the gamma ray shield portion 14 is formed of lead, the first neutron absorption portion 15 is formed of rubber containing boron carbide, and the second neutron absorption portion 16 is formed of lithium fluoride.

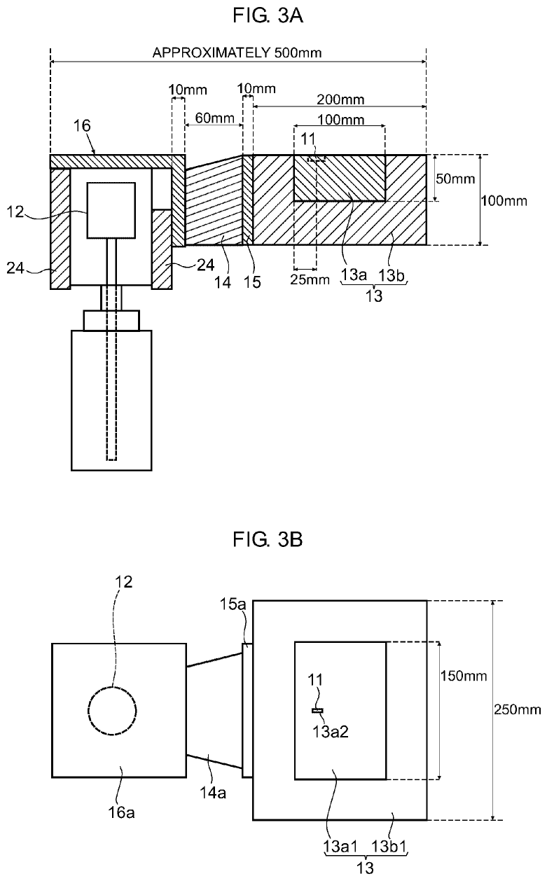

FIG. 3A and FIG. 3B correspond to FIG. 1A and FIG. 1B, respectively, and illustrate dimensions of respective parts in the non-destructive inspection device 10 of the implementation example. As illustrated in FIG. 3A and FIG. 3B, the non-destructive inspection device 10 of the implementation example is configured to have a small lateral-direction dimension of approximately 500 mm. In the non-destructive inspection device 10 of the implementation example, a total weight of the neutron source 11, the gamma ray detector 12, the neutron shield portion 13, the gamma ray shield portion 14, the first neutron absorption portion 15, the second neutron absorption portion 16, the liquid nitrogen dewar 17, and the cold finger 18 is smaller than 30 kg. The sum of this the total weight and a weight of the sub-detector 24 is smaller than 50 kg.

When chlorine as the target component having a concentration of 3.0 kg/m³ exists at a depth in a range of 0 cm to 3 cm from the surface 1a in the inspection object 1 as the concrete structure, the non-destructive inspection device 10 of the implementation example was able to detect the existence of chlorine and the concentration of chlorine (³⁵Cl).

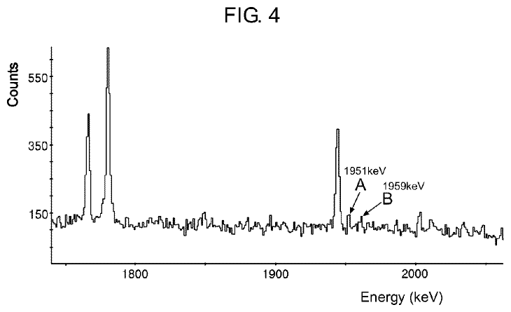

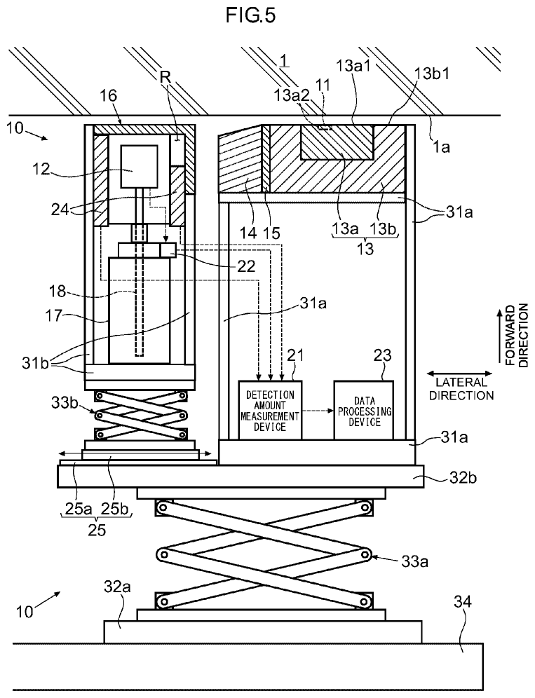

FIG. 4 illustrates the energy spectrum of the gamma rays acquired by the detection amount measurement device 21 in that case. In FIG. 4, the horizontal axis indicates energy of the gamma ray, and the vertical axis indicates a detection amount of the gamma rays (the number of times of detecting the gamma ray). In FIG. 4, the arrows A and B each indicate the number of counts of the gamma ray derived from the isotope ³⁵Cl and having energy of 1,951 keV or 1,959 keV. Based on this energy spectrum, the above-mentioned concentration of chlorine at a depth in a range of 0 cm to 3 cm was determined by the data processing device 23.

The present invention is not limited to the above-described embodiment, and it is apparent that various modifications may be made within the scope of the technical idea of the present invention. For example, the non-destructive inspection device 10 according to the embodiment of the present invention does not need to include all of the above-described plurality of matters, and may include only a part of the above-described plurality of matters. For example, one or both of the sub-detector 24 and the front absorption portion 16a may be omitted.

Any one of the below-described modified examples 1 to 5 may be adopted alone, or two or more of the modified examples 1 to 5 may be appropriately adopted in combination. In this case, the matters that are not described below are the same as those described above.

### Modified Example 1

FIG. 5 illustrates a configuration of a non-destructive inspection device 10 according to the modified example 1. In the above-described embodiment, the neutron source 11, the neutron shield portion 13, the gamma ray shield portion 14, the gamma ray detector 12, and the like are integrally movable in the direction parallel to the forward direction. However, the following configuration may be adopted. Here, a first structure is defined as a structure including the neutron source 11, the neutron shield portion 13, and the gamma ray shield portion 14, and a second structure is defined as a structure including the gamma ray detector 12. One (in the example of FIG. 5, the second structure including the gamma ray detector 12) of the first and second structures may be movable relative to the other of the first and second structures in at least one of the direction parallel to the forward direction and the lateral direction. In other words, the non-destructive inspection device 10 may further include at least one (in the example of FIG. 5, both) of the below-described second position adjustment mechanism 33b for the direction parallel to the forward direction and the below-described distance adjustment mechanism 25 for the lateral direction.

In present modified example 1, first and second supports 31a and 31b are provided instead of the above-described support 31. The first support 31a supports the neutron shield portion 13, the gamma ray shield portion 14, and the first neutron absorption portion 15. The neutron shield portion 13, the gamma ray shield portion 14, and the first neutron absorption portion 15 may be integrated with each other by being attached to the first support 31a. The second support 31b supports the gamma ray detector 12 and the second neutron absorption portion 16 (and the dewar 17 and the sub-detector 24). The gamma ray detector 12 and the second neutron absorption portion 16 (and the dewar 17 and the sub-detector 24) may be integrated with each other by being attached to the second support 31b.

In present modified example 1, the non-destructive inspection device 10 includes the above-described base 32 and position adjustment mechanism 33 as a first base 32a and a first position adjustment mechanism 33a, and further includes a second base 32b. The first base 32a supports the second base 32b via the first position adjustment mechanism 33a.

### <Second Position Adjustment Mechanism>

The non-destructive inspection device 10 may further include the second base 32b and the second position adjustment mechanism 33b. In this case, the second base 32b supports one (in the example of FIG. 5, the second support 31b) of the first and second supports 31a and 31b via the second position adjustment mechanism 33b, and directly supports the other of the first and second supports 31a and 31b.

The second position adjustment mechanism 33b supports one of the first and second supports 31a and 31b, and enables a position of the one of the first and second supports 31a and 31b to be adjusted relative to the other of the first and second supports 31a and 31b in the direction parallel to the forward direction. Thereby, the second position adjustment mechanism 33b enables a position of one of the first structure including the neutron shield portion 13 and the second structure including the gamma ray detector 12 to be adjusted in the direction parallel to the forward direction, relative to the other of the first structure including the neutron shield portion 13 and the second structure including the gamma ray detector 12. The second position adjustment mechanism 33b may be configured by a jack similarly to the position adjustment mechanism 33 in the above-described embodiment, or may have any of other configurations.

### <Distance Adjustment Mechanism>

The non-destructive inspection device 10 may include the distance adjustment mechanism 25 that allows a lateral-direction distance from the gamma ray detector 12 to the neutron shield portion 13 and the gamma ray shield portion 14 to be adjusted. The distance adjustment mechanism 25 may be provided on the second base 32b.

The distance adjustment mechanism 25 may be constituted by a stage or a slide mechanism that is movable in the lateral direction. As illustrated in FIG. 5B, such a distance adjustment mechanism 25 may include a guide mechanism 25a (e.g., a rail) and a movable portion 25b (e.g., a stage). The guide mechanism 25a is provided on the second base 32b and guides a lateral-direction movement of the movable portion 25b relative to the second base 32b. The movable portion 25b is configured to be movable in the lateral direction relative to the second base 32b while being supported by the second base 32b (e.g., via the guide mechanism 25a).

One (in the example of FIG. 5, the second support 31b) of the first and second supports 31a and 31b is attached to the movable portion 25b. Here, when the one of the first and second supports 31a and 31b is supported by the second base 32b via the second position adjustment mechanism 33b, the one is attached to the movable portion 25b via the second position adjustment mechanism 33b. In the example of FIG. 5B, the second support 31b is attached to the movable portion 25b via the second position adjustment mechanism 33b.

Such a configuration allows the movable portion 25b to slide in the lateral direction manually or by an appropriate driving device to adjust a lateral-direction distance between the gamma ray detector 12 and the neutron shield portion 13. After this adjustment, a position of the movable portion 25b may be held by an appropriate stopper or locking mechanism.

In the modified example 1, the first position adjustment mechanism 33a may be omitted. In this case, for example, the first base 32a may be further omitted, and the second base 32b may be directly installed on the bed 34.

### Modified Example 2

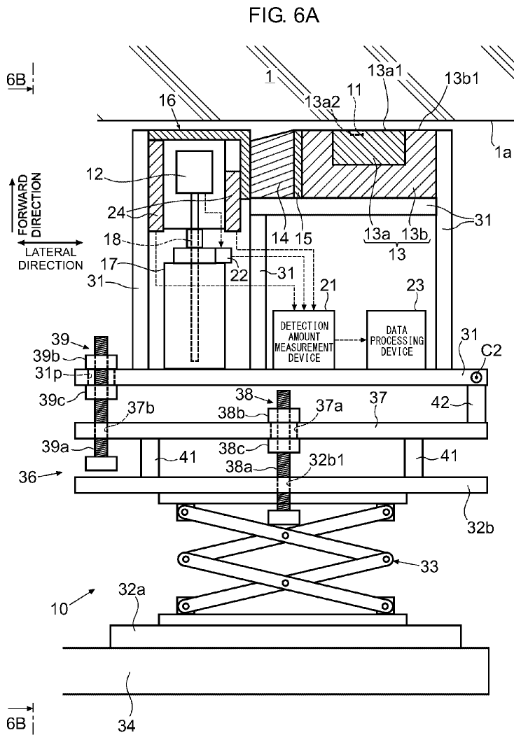

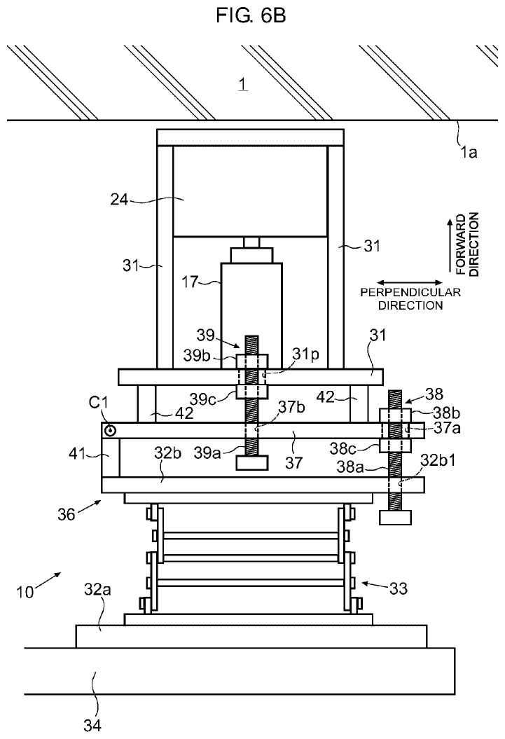

A non-destructive inspection device 10 according to the present modified example 2 further includes an inclination adjustment mechanism in addition to the configuration of the above-described embodiment. The inclination adjustment mechanism allows an inclination of the neutron shield portion 13, the gamma ray shield portion 14, and the gamma ray detector 12 to be adjusted to a front side or a rear side. Configuration examples of such an inclination adjustment mechanism 36 are described with reference to FIG. 6A to FIG. 9C. The sub-detector 24 may be omitted in FIG. 6A to FIG. 9C.

In each of the configuration examples illustrated in FIG. 6A to FIG. 9C, the non-destructive inspection device 10 according to the modified example 2 may include the above-described base 32 as the first base 32a, and may further include the second base 32b. The first base 32a supports the second base 32b via the above-described position adjustment mechanism 33, and the second base 32b supports the support 31 via the inclination adjustment mechanism 36. A position of the second base 32b relative to the first base 32a is adjustable in the direction parallel to the forward direction by the position adjustment mechanism 33. An inclination of the support 31 toward a front side or a rear side (an inclination relative to the second base 32b) is adjustable by the inclination adjustment mechanism 36. In other words, the inclination adjustment mechanism 36 enables an inclination of the support 31 to be adjusted in such a way as to shift one end portion of the support 31 relative to an opposite end portion of the support 31 in the forward direction or the rearward direction. Here, this one end portion of the support 31 is one end portion in a predetermined direction (e.g., the lateral direction) perpendicular to the forward direction, and this opposite end portion of the support 31 is located on an opposite side of this one end portion in this predetermined direction.

In this manner, the inclination adjustment mechanism 36 enables adjustment of a front-side or rear-side inclination of the neutron shield portion 13, the gamma ray shield portion 14, and the gamma ray detector 12 that are supported by the support 31.

### Configuration Example 1

FIG. 6A illustrates an example of the non-destructive inspection device 10 including the inclination adjustment mechanism 36 according to the configuration example 1. FIG. 6B is a view in the arrows 6B-6B in FIG. 6A. The inclination adjustment mechanism 36 allows the support 31 to rotate around each of first and second axes C1 and C2, and thereby allows an inclination of the support 31 to be adjusted to a front side or a rear side. The axis C1 is oriented in the lateral direction, and the axis C2 is oriented in a direction (hereinafter, also referred to simply as the perpendicular direction) perpendicular to both of the forward direction and the lateral direction. The inclination adjustment mechanism 36 includes a swing body 37 and first and second adjustment units 38 and 39.

As illustrated in FIG. 6B, the swing body 37 is supported by the second base 32b via a first support member 41 in such a way as to be swingable around the first axis C1 relative to the second base 32b. As illustrated in FIG. 6A, the support 31 is supported by the second base 32b via a second support member 42 in such a way as to be swingable around the second axis C2 relative to the swing body 37.

The first adjustment unit 38 includes a bolt 38a and first and second nuts 38b and 38c. With respect to the bolt 38a, a screw hole 32b1 penetrating the second base 32b in the forward direction is provided in the second base 32b, and a penetration hole 37a penetrating the swing body 37 in the forward direction is provided in the swing body 37. The bolt 38a is arranged to penetrate the penetration hole 37a while being screwed into the screw hole 32b1. The first and second nuts 38b and 38c are screwed onto the bolt 38a in such a way as to sandwich the swing body 37 in the forward direction. Rotating the first and second nuts 38b and 38c can cause the swing body 37 to swing around the first axis C1 relative to the second base 32b. For example, the first nut 38b is loosened and the second nut 38c is rotated to move to a front side or a rear side to adjust an inclination of the swing body 37 around the first axis C1, and then, the first nut 38b is tightened to the swing body 37 to fix the inclination of the swing body 37. A perpendicular-direction dimension of the penetration hole 37a is longer than a cross-sectional dimension of the bolt 38a so that the swing body 37 can be inclined relative to the second base 32b.

Similarly, the second adjustment unit 39 includes a bolt 39a and first and second nuts 39b and 39c. With respect to the bolt 39a, a screw hole 37b penetrating the swing body 37 in the forward direction is provided in the swing body 37, and a penetration hole 31p penetrating the support 31 in the forward direction is provided in the support 31. The bolt 39a is arranged to penetrate the penetration hole 31p while being screwed into the screw hole 37b. The first and second nuts 39b and 39c are screwed onto the bolt 39a in such a way as to sandwich the support 31 in the forward direction. Similarly to the case of the first adjustment unit 38, the support 31 is swingable around the second axis C2 relative to the swing body 37 by rotating the first and second nuts 39b and 39c. A lateral-direction dimension of the penetration hole 31p is longer than a cross-sectional dimension of the bolt 39a so that the support 31 can be inclined relative to the swing body 37.

When the forward direction is the vertically upward direction, the first nuts 38b and 39b may be omitted in the first and second adjustment units 38 and 39.

### Configuration Example 2

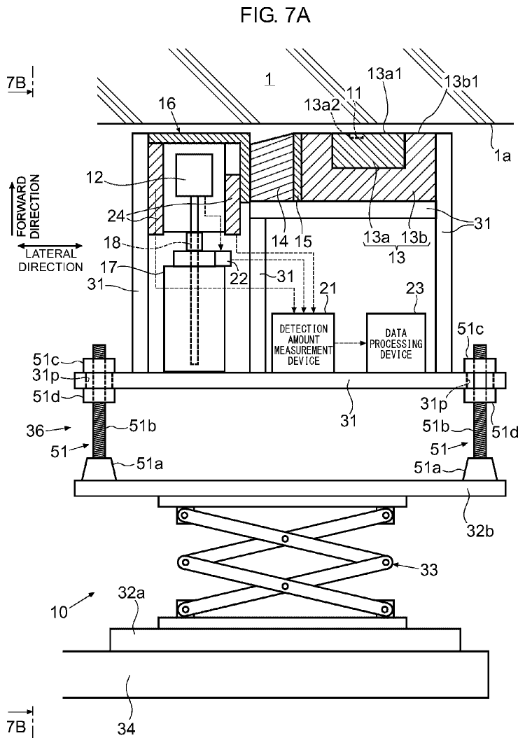

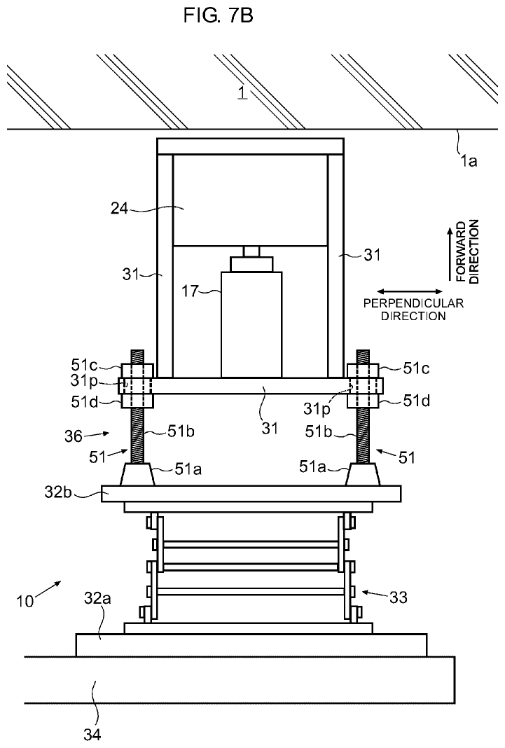

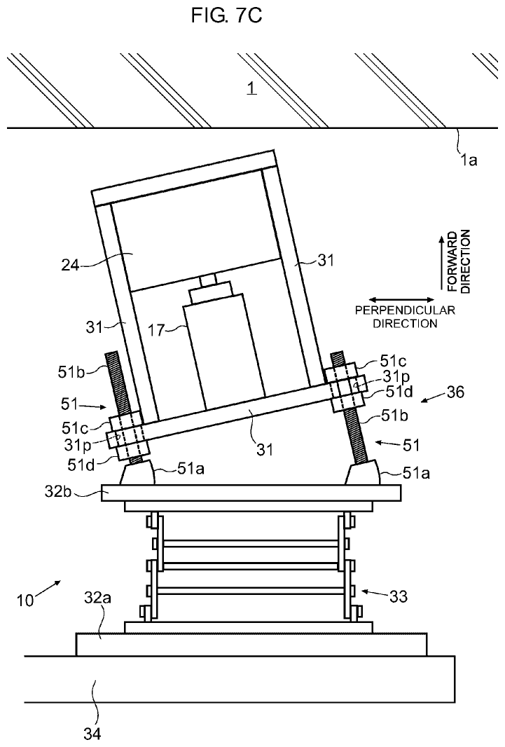

FIG. 7A illustrates an example of the non-destructive inspection device 10 including the inclination adjustment mechanism 36 according to the configuration example 2. FIG. 7B is a view in the arrows 7B-7B in FIG. 7A. The inclination adjustment mechanism 36 includes a plurality of (in the example of the drawings, four) connection units 51 that are arranged to be separated from each other when viewed from a front side.

Each of the connection units 51 includes an attachment member 51a, a bolt 51b, and first and second nuts 51c and 51d. The attachment member 51a is attached to the second base 32b. One end portion of the bolt 51b is coupled to the attachment member 51a. With respect to the bolt 51b, a penetration hole 31p penetrating the support 31 in the forward direction is formed in the support 31. The bolt 51b extends from one end portion to a front side to penetrate the penetration hole 31p. The first and second nuts 51c and 51d are screwed onto the bolt 51b in such a way as to sandwich the support 31 in the forward direction.

The support 31 can be inclined to a front side or a rear side by rotating the first and second nuts 51c and 51d in each of the connection units 51, as illustrated in FIG. 7C, for example. Such inclination may be enabled by elastic deformation of the attachment members 51a as illustrated in FIG. 7C, or may be enabled by swingably coupling one end portion of the bolt 51b to the attachment member 51a (by a ball joint, for example). A cross-sectional dimension of the penetration hole 31p is sufficiently larger than a cross-sectional dimension of the bolt 51b so that the support 31 can be inclined relative to the second base 32b. However, a cross-sectional dimension or a shape of the penetration hole 31p is set in such a way as to prevent the first and second nuts 51c and 51d from passing through the penetration hole 31p. When the forward direction is the vertically upward direction, the first nut 51c may be omitted.

### Configuration Example 3

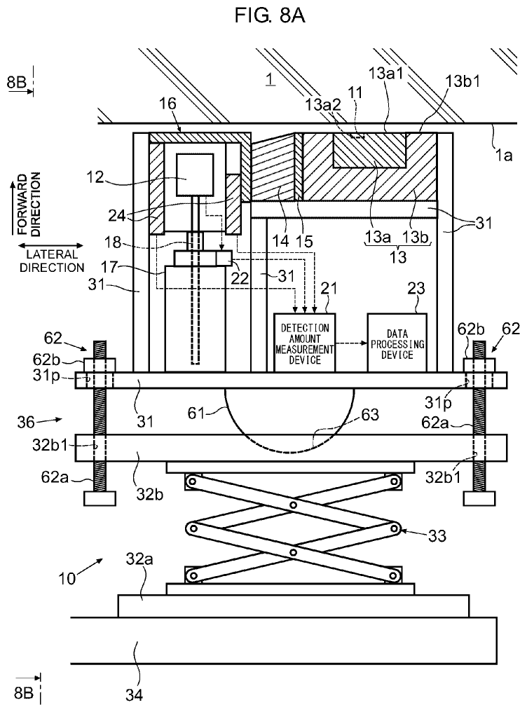

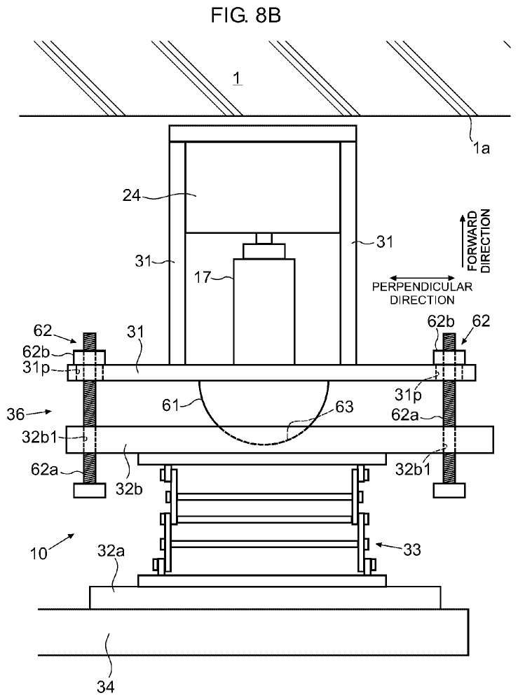

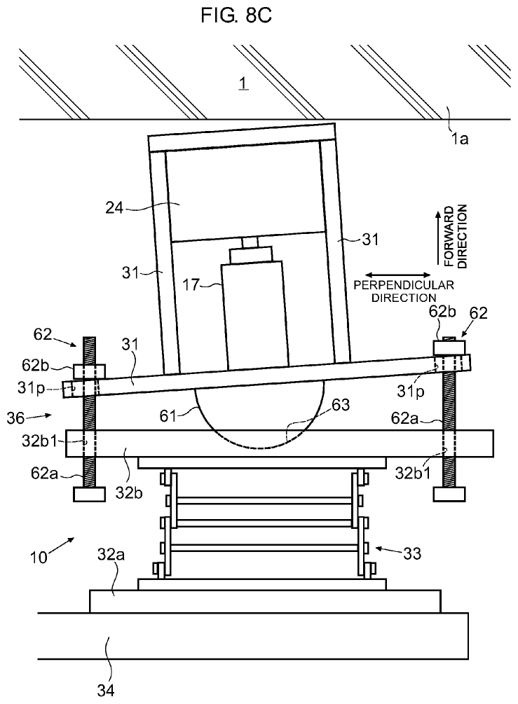

FIG. 8A illustrates an example of the non-destructive inspection device 10 including the inclination adjustment mechanism 36 according to the configuration example 3. FIG. 8B is a view in the arrows 8B-8B in FIG. 8A. The inclination adjustment mechanism 36 includes a spherical-surface portion 61 and a plurality of adjustment units 62.

The spherical-surface portion 61 has a spherical crown shape (e.g., a hemispherical shape) formed by cutting a sphere with a plane. A bottom surface (i.e., the cut surface in the spherical crown shape) of the spherical-surface portion 61 is attached to one (in FIG. 8A, the support 31) of a lower surface of the support 31 and an upper surface of the second base 32b. A part of a spherical surface of the spherical-surface portion 61 is received in a recess 63 provided on the other of the lower surface of the support 31 and the upper surface of the second base 32b. An inner surface of the recess 63 has a shape that matches with the spherical surface of the spherical-surface portion 61.

A plurality of the adjustment units 62 are arranged to be separated from each other when viewed from a front side. In the example of FIG. 8A and FIG. 8B, the four adjustment units 62 are provided.

Each of the adjustment units 62 includes a bolt 62a and a nut 62b. With respect to the bolt 62a, a screw hole 32b1 penetrating the second base 32b in the forward direction is provided in the second base 32b, and a penetration hole 31p penetrating the support 31 in the forward direction is provided in the support 31. The bolt 62a is arranged to penetrate the penetration hole 31p while being screwed into the screw hole 32b1. The nut 62b is screwed onto the bolt 62a, on a front side of the support 31.

Such a configuration enables rotation amounts of the nuts 62b of a plurality of the adjustment units 62 to be adjusted, so that the spherical surface of the spherical-surface portion 61 slides on an inner surface of the recess 63. Thereby, as illustrated in FIG. 8C, an inclination of the support 31 is adjusted to a front side or a rear side. A cross-sectional dimension of the penetration hole 31p is sufficiently larger than a cross-sectional dimension of the bolt 62a so that the support 31 can be inclined relative to the second base 32b. However, a cross-sectional dimension or a shape of the penetration hole 31p is set in such a way as to prevent the nut 62b from passing through the penetration hole 31p.

### Configuration Example 4

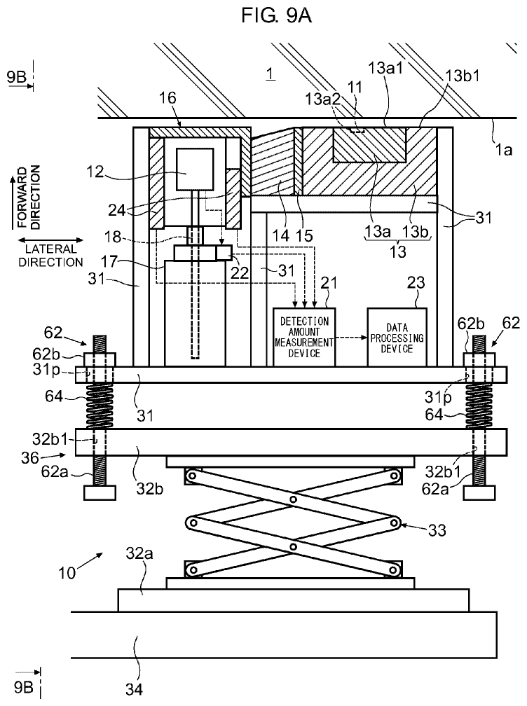

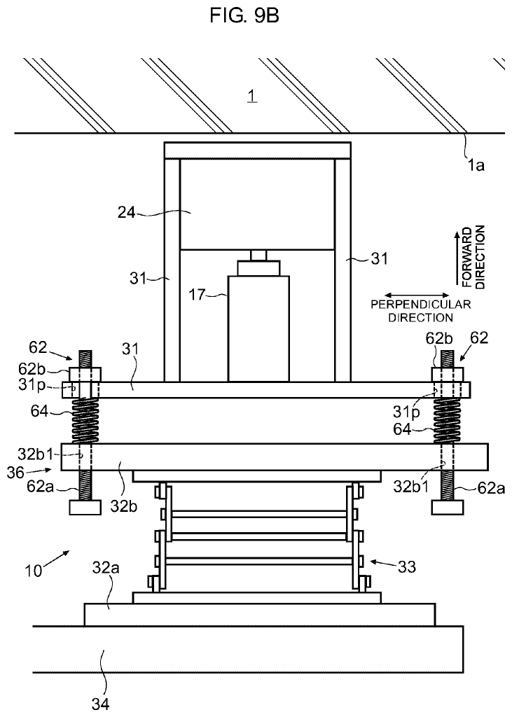

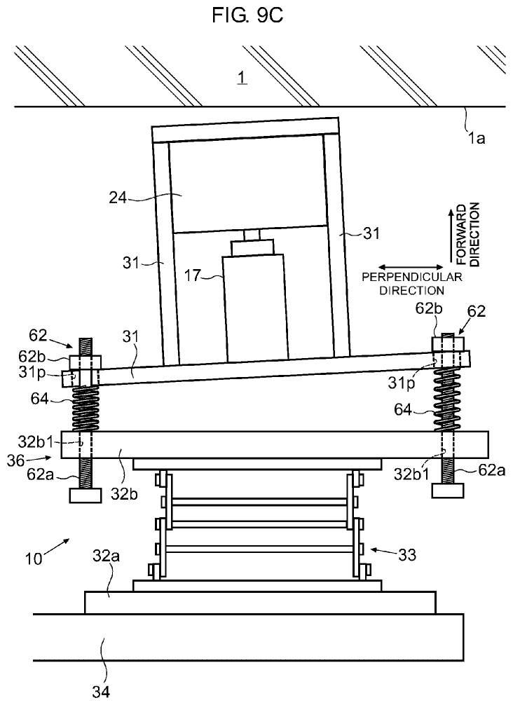

FIG. 9A illustrates an example of the non-destructive inspection device 10 including the inclination adjustment mechanism 36 according to the configuration example 4. FIG. 9B is a view in the arrows 9B-9B in FIG. 9A. The inclination adjustment mechanism 36 includes a plurality of coil-shaped springs 64 instead of the spherical-surface portion 61 of the inclination adjustment mechanism 36 according to the configuration example 3, and the other configurations thereof are the same as those of the inclination adjustment mechanism 36 according to the configuration example 3.

In each of the adjustment units 62, the bolt 62a is arranged inside the spring 64. The bolt 62a penetrates the spring 64 in the axial direction of the spring 64. The spring 64 is in a compressed state between the support 31 and the second base 32b, and biases the support 31 and the second base 32b in a direction of separating the support 31 and the second base 32b from each other.

Such a configuration enables rotation amounts of the nuts 62b of a plurality of the adjustment units 62 to be adjusted so that compression amounts of the associated springs 64 can be changed. Thereby, an inclination of the support 31 is adjusted to a front side or a rear side, as illustrated in FIG. 9C.

The inclination adjustment mechanism 36 is not limited to the above-described configuration examples 1 to 4, and may have any of other configurations.

### Modified Example 3

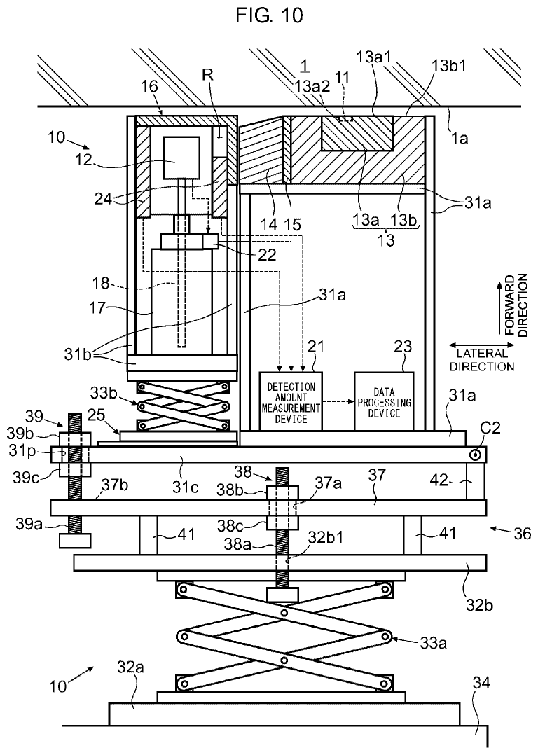

In the non-destructive inspection device 10 of the modified example 1, the inclination adjustment mechanism of the modified example 2 may be provided as described below. FIG. 10 illustrates a configuration example in this case. FIG. 10 illustrates a case where the inclination adjustment mechanism 36 of the configuration example 1 in the modified example 2 is provided in the non-destructive inspection device 10 of the modified example 1. Hereinafter, the second base 32b corresponds to the second base 32b in the above-described modified example 2, and a third support 31c corresponds to the second base 32b in the modified example 1 and corresponds to the support 31 in the modified example 2.

With such a configuration, the first position adjustment mechanism 33a enables positions of the gamma ray detector 12, the neutron shield portion 13, the gamma ray shield portion 14, and the like (i.e., the first to third supports 31a to 31c) to be adjusted relative to the first base 32a in the direction parallel to the forward direction. In addition, one or both of the second position adjustment mechanism 33b and the distance adjustment mechanism 25 enables a position of one of the first structure (the first support 31a) including the neutron shield portion 13 and the gamma ray shield portion 14 and the second structure (the second support 31b) including the gamma ray detector 12 to be adjusted relative to a position of the other of the first and second structures (the first and second supports 31a and 31b) in one or both of the direction parallel to the forward direction and the lateral direction. Further, the inclination adjustment mechanism 36 enables an inclination of the gamma ray detector 12, the neutron shield portion 13, and the like (i.e., the first to third support 31a to 31c) to be adjusted to a front side or a rear side.

### Modified Example 4

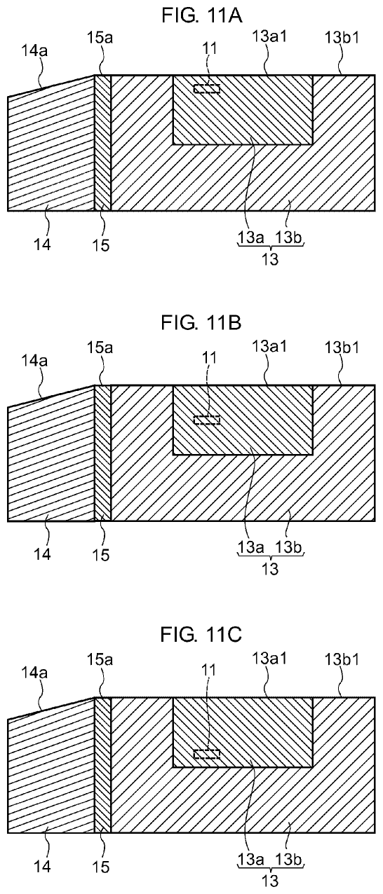

The neutron source 11 may be arranged inside the deceleration portion 13a in such a way that the deceleration portion 13a covers the neutron source 11 not only from an area around and a rear side of the neutron source 11 but also from a front side of the neutron source 11. FIG. 11A to FIG. 11C illustrate arrangement examples of the neutron source 11 in the modified example 4. FIG. 11A to FIG. 11C correspond to an enlarged partial view of FIG. 1A, and each illustrate the neutron source 11 whose position is different from that in FIG. 1A.

As illustrated in FIG. 11A, the neutron source 11 may be located on a front side of a depth-direction center of the deceleration portion 13a. Alternatively, as illustrated in FIG. 11B, the neutron source 11 may be located at the depth-direction center of the deceleration portion 13a. Alternatively, as illustrated in FIG. 11C, the neutron source 11 may be located on a rear side of the depth-direction center of the deceleration portion 13a. The depth-direction center of the deceleration portion 13a may mean a center of the deceleration portion 13a in a direction perpendicular to the neutron emission surface 13a1.

Arranging the neutron source 11 inside (e.g., at a deep position in) the deceleration portion 13a in such a manner causes neutrons from the neutron source 11 to be scattered and decelerated in the deceleration portion 13a. Thus, more thermal neutrons among the neutrons from the neutron source 11 can enter the inspection object 1. Such arrangement can increase an amount of gamma rays generated by reaction between the thermal neutrons and the target component (e.g., chlorine) in the inspection object 1 when an amount of hydrogen elements in the inspection object 1 is small. Further, such arrangement can increase an amount of gamma rays generated by reaction between the thermal neutron and the target component (e.g., chlorine) near the surface 1a (e.g., at a depth of approximately 3 cm from the surface 1a) in the same inspection object 1.

### Modified Example 5

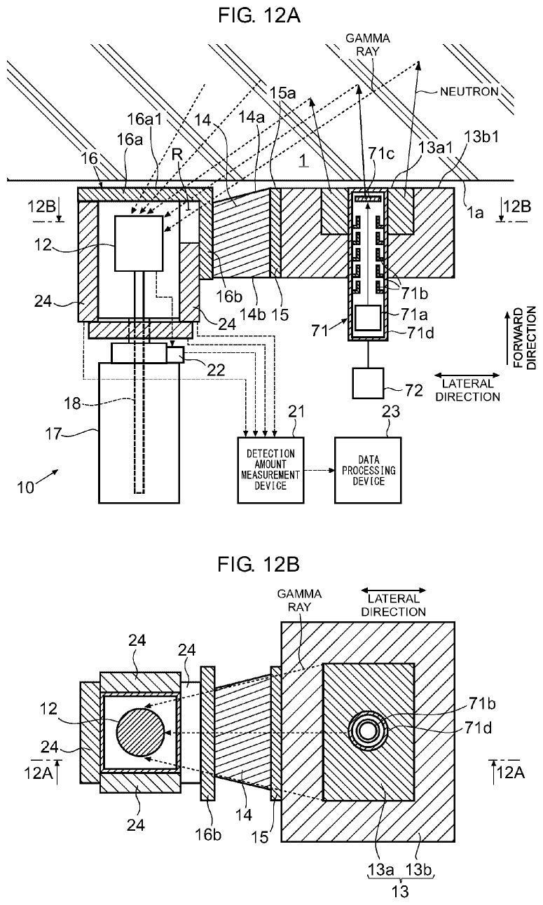

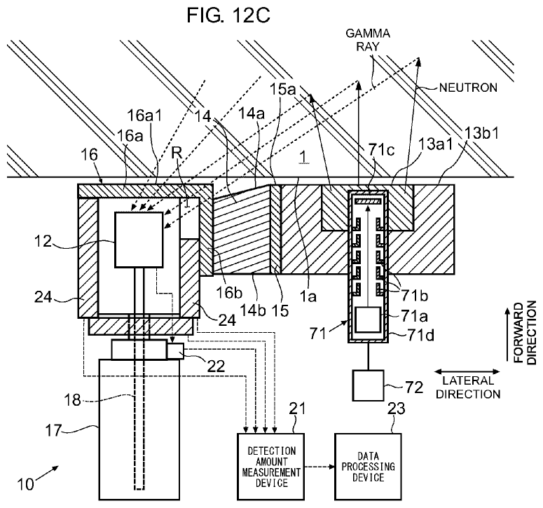

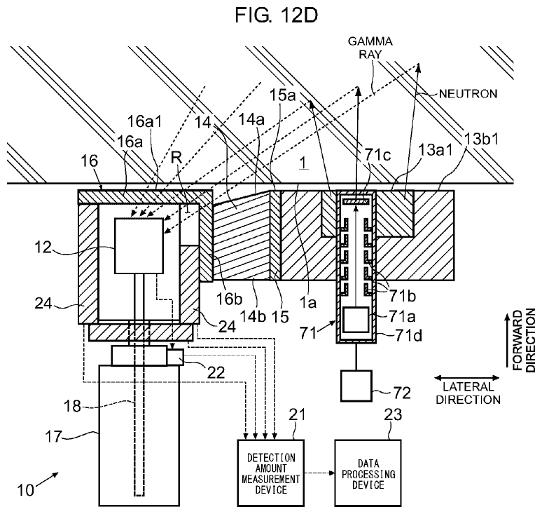

Instead of the above-described neutron source 11, a neutron generation tube 71 may be provided. A configuration example in this case is illustrated in FIG. 12A. FIG. 12B is a horizontal sectional view taken along line 12B-12B in FIG. 12A. FIG. 12A is a sectional view taken along line 12A-12A in FIG. 12B.

The neutron generation tube 71 includes an ion source 71a, an accelerator 71b, a target 71c that serves as the neutron generation portion, and a tubular receptacle 71d. The ion source 71a generates deuterium ions or tritium ions. The accelerator 71b (acceleration electrodes) accelerates the deuterium ions or the tritium ions from the ion source 71a toward the target 71c. The target 71c occludes deuterium or tritium. The receptacle 71d has a tubular shape extending in the forward direction. The ion source 71a, the accelerator 71b, and the target 71c are arranged inside the receptacle 71d.

In such a configuration, the deuterium ions or the tritium ions accelerated as described above collide with the target 71c, and thereby, neutrons are generated from the target 71c. In other words, the neutrons are generated by nuclear fusion reaction between the tritium or deuterium occluded in the target 71c and the collided deuterium ions or tritium ions. This nuclear fusion reaction is DD nuclear fusion reaction between deuterium (D) and deuterium (D) or DT nuclear fusion reaction between deuterium (D) and tritium (T). The target 71c constitutes the neutron generation portion of the present invention.

A structure of such a neutron generation tube 71 itself is known, and thus, detailed description thereof is omitted. A power supply device 72 that supplies a voltage to each part of the neutron generation tube 71 may be provided for operating the neutron generation tube 71. The power supply device 72 may be supported, on a rear side of the neutron shield portion 13, by the above-described support 31.

According to the modified example 5, the target 71c is arranged inside a front end portion of the neutron generation tube 71. In other words, the target 71c is arranged inside a forward-direction front end portion of the receptacle 71d. The neutron shield portion 13 covers the front end portion of the neutron generation tube 71 (i.e., the forward-direction front end portion of the receptacle 71d) from an area around this front end portion. More specifically, the deceleration portion 13a of the neutron shield portion 13 covers the front end portion (the forward-direction front end portion of the receptacle 71d) of the neutron generation tube 71 from the area around this front end portion, and the reflection portion 13b of the neutron shield portion 13 covers the deceleration portion 13a from an area around and a rear side of the deceleration portion 13a. The receptacle 71d may extend in the rearward direction from the forward-direction front end portion thereof as far as a position on a rear side of the neutron shield portion 13.

A front end surface of the receptacle 71d in the forward direction may be exposed to an outside as illustrated in FIG. 12A, or may be covered with the deceleration portion 13a from a front side as illustrated in FIG. 12C. When viewed in the rearward direction, the receptacle 71d (i.e., the target 71c) may be arranged at a lateral-direction center of the deceleration portion 13a as illustrated in FIG. 12B, or may be arranged at a position shifted toward the gamma ray detector 12 from the lateral-direction center of the deceleration portion 13a as illustrated in FIG. 12D.

### (Features of Non-Destructive Inspection Device and Advantageous Effects)

The non-destructive inspection device according to the above-described embodiment and the modified examples 1 to 5 may also be described as follows.

### Supplementary Note 1

A non-destructive inspection device including:
a neutron generation portion configured to spontaneously generate and emit neutrons or to generate and emit neutrons by DD nuclear fusion reaction or DT nuclear fusion reaction;
a neutron shield portion configured to cover the neutron generation portion from at least an area around the neutron generation portion and thereby shield the neutrons at the area around the neutron generation portion, and allow the neutrons to be emitted to a front side of the neutron generation portion; and
a gamma ray detector configured to detect gamma rays generated in an inspection object on a front side of the neutron generation portion, and output a detection signal concerning the detection, the gamma rays being generated as a result of the neutrons incident on the inspection object, wherein
the neutron shield portion and the gamma ray detector are arranged in alignment with each other in a lateral direction in relation to a forward direction, the forward direction being a direction from a rear side of the neutron generation portion to a front side of the neutron generation portion.

### Advantageous Effect of Supplementary Note 1

When the neutron generation portion spontaneously generates and emits neutrons, large-scale device and accelerator that generate a charged particle beam is unnecessary differently from a neutron source that causes the charged particle beam to collide with a target to generate the neutrons. Thus, the non-destructive inspection device can be reduced in a size and a weight so that the non-destructive inspection device can be easily transported and arranged.

When the neutron generation portion generates and emits neutrons by the DD nuclear fusion reaction or the DT nuclear fusion reaction, a high reaction cross-section can be achieved at a low acceleration voltage for generating the neutrons. For this reason, a large-scale accelerator or the like is unnecessary. Thus, the non-destructive inspection device can be reduced in a size and a weight so that the non-destructive inspection device can be easily transported and arranged.

The neutron shield portion that covers the neutron generation portion from at least the area around the neutron generation portion, and the gamma ray detector are arranged in alignment with each other in the lateral direction. Accordingly, both the neutron generation portion surrounded by the neutron shield portion and the gamma ray detector can be arranged in the vicinity of a surface of the inspection object. Thus, the neutrons from the neutron generation portion can be efficiently made incident on the inspection object, and at the same time, the gamma rays generated in the inspection object as a result of the neutrons can be efficiently detected. For this reason, a sufficient detection amount of the gamma rays can be ensured even in a case of the neutron generation portion (e.g., the above-described RI neutron source such as ²⁵²Cf or the above-described RI neutron generation tube) that emits neutrons whose amount is small as compared with a neutron source using a large-scale device and accelerator whose transportation and arrangement is difficult and that cause a charged particle beam to collide with a target and thereby generate neutrons. Thus, presence or absence, a depth, or a concentration of the target component in the inspection object can be determined based on the detected gamma rays.

### Supplementary Note 2

The non-destructive inspection device according to Supplementary Note 1, wherein
the neutron generation portion is a neutron source configured to spontaneously generate and emit neutrons, and
the neutron shield portion is configured to cover the neutron source from the area around and a rear side of the neutron source and shield the neutrons at the area around and the rear side of the neutron source, and allow the neutrons to be emitted to the front side of the neutron source.

### Advantageous Effect of Supplementary Note 2

According to this configuration, the neutron generation portion is the neutron source spontaneously generating and emitting neutrons, and thus, does not need an ion source, an accelerator, and the like for the generation of the neutrons. Thus, the neutron shield portion can easily cover the neutron generation portion, and can cover the neutron source also from a rear side of the neutron source.

### Supplementary Note 3

The non-destructive inspection device according to Supplementary Note 1, further including:
a neutron generation tube configured to generate the neutrons by the DD nuclear fusion reaction or the DT nuclear fusion reaction, wherein
the neutron generation portion is a target that is arranged inside a front end portion of the neutron generation tube and that generates the neutrons by the DD nuclear fusion reaction or the DT nuclear fusion reaction caused by deuterium ions or tritium ions colliding with the target, and
the neutron shield portion covers the front end portion of the neutron generation tube and the target from the area around the front end portion.

### Advantageous Effect of Supplementary Note 3

As described above, the neutron generation tube does not need a large-scale accelerator and the like. Thus, using the neutron generation tube instead of the neutron source spontaneously generating neutrons enables implementation of the non-destructive inspection device that can be easily transported and arranged and that enables detection of a target component in the inspection object.

### Supplementary Note 4

The non-destructive inspection device according to any one of Supplementary Notes 1 to 3, wherein
the neutron shield portion includes
   a deceleration portion that is formed of a material decelerating the neutrons and that covers the neutron generation portion from at least the area around the neutron generation portion, and
   a reflection portion that is formed of a material reflecting the neutrons and that covers the deceleration portion from an area around and a rear side of the deceleration portion.

### Advantageous Effect of Supplementary Note 4

According to this configuration, fast neutrons emitted from the neutron generation portion to the area around the neutron generation portion can make at least one round trip of advancing to the reflection portion via the deceleration portion and being then reflected by the reflection portion and thereby returning to the deceleration portion, and then advance to the inspection object on a front side. Thereby, even when an amount of neutrons from the neutron source is relatively small, more neutrons can be caused to enter the inspection object.

Such neutrons are scattered and decelerated in the deceleration portion, and thus, more neutrons as thermal neutrons can enter the inspection object. For example, a generation amount of gamma rays derived from chlorine in the inspection object increases by reaction between chlorine and the thermal neutrons having low neutron energy. Accordingly, more thermal neutrons incident on the inspection object can increase a detection amount of the gamma rays derived from chlorine as the target component.

### Supplementary Note 5

The non-destructive inspection device according to Supplementary Note 2, wherein
the neutron shield portion includes
   a deceleration portion that is formed of a material decelerating the neutrons and that covers the neutron source from the area around and a rear side of the neutron source, and
   a reflection portion that is formed of a material reflecting the neutrons and that covers the deceleration portion from an area around and a rear side of the deceleration portion, and
the neutron source is arranged at a front end portion of the deceleration portion in the forward direction.

### Advantageous Effect of Supplementary Note 5

According to this configuration, the neutrons emitted to a front side from the neutron source enter the inspection object without passing through the deceleration portion, without being decelerated by the deceleration portion, and without being reflected by the reflection portion at the area around and the rear side of the deceleration portion. This can suppress an amount of neutrons that are generated from the neutron source and that are attenuated in the deceleration portion. The neutrons entering the inspection object (e.g., without passing through the deceleration portion) as the fast neutrons among the neutrons from the neutron source are decelerated in the inspection object (e.g., hydrogen element) and become the thermal neutrons. Thus, more neutrons from the neutron source can become thermal neutrons in the inspection object, thereby additionally increasing the number of thermal neutrons in the inspection object. As a result, for example, an amount of gamma rays derived from chlorine as the target component that easily reacts with the thermal neutrons can be increased. Meanwhile, in a case of an inspection object containing a small amount of hydrogen elements or an inspection object having a large volume, the above-described modified example 4 is preferably adopted in some cases.

### Supplementary Note 6

The non-destructive inspection device according to Supplementary Note 2, wherein
the neutron shield portion includes
   a deceleration portion that is formed of a material decelerating the neutrons and that covers the neutron source from the area around and a rear side of the neutron source, and
   a reflection portion that is formed of a material reflecting the neutrons and that covers the deceleration portion from an area around and a rear side of the deceleration portion, and
the neutron source is arranged at a position shifted toward the gamma ray detector from a lateral-direction center of the deceleration portion when viewed in a direction opposite to the forward direction.

### Advantageous Effect of Supplementary Note 6

With this configuration, a distance between the neutron source and the gamma ray detector is reduced. As a result, it was confirmed from the experiment and the radiation transport simulation that an amount (the number of times of detecting the gamma ray) of the gamma rays detected by the gamma ray detector was increased.

### Supplementary Note 7

The non-destructive inspection device according to Supplementary Note 4, wherein the deceleration portion includes a neutron emission surface that faces in the forward direction and that emits the neutrons from the neutron generation portion.

### Advantageous Effect of Supplementary Note 7

In this manner, the front surface included in the deceleration portion and facing in the forward direction functions as the neutron emission surface. When the neutron generation portion is the above-described neutron source that spontaneously generates and emits neutrons, even when the neutron source is small, the neutron emission surface is a wide area having a spread surrounding the neutron source as a local narrow area when viewed in a direction opposite to the forward direction. Such a neutron emission surface emits the neutrons to an outside. In this case, the neutrons emitted from the neutron source to the area around and the rear side of the neutron source can be reflected by the reflection portion once or a plurality of times, and can be then emitted from the neutron emission surface. In the example, in FIG. 3A and FIG. 3B, a perpendicular-direction dimension and a lateral-direction dimension of the neutron emission surface 13a1 are approximately 150 mm and approximately 100 mm, respectively, and meanwhile, a perpendicular-direction dimension and a lateral-direction dimension of the neutron source 11 are approximately 10 mm and approximately 35 mm, respectively. However, the present invention is not limited to these numerical values.

### Supplementary Note 8

The non-destructive inspection device according to Supplementary Note 4, further including:
a gamma ray shield portion that is arranged between the neutron shield portion and the gamma ray detector in the lateral direction and that shields gamma rays.

### Advantageous Effect of Supplementary Note 8

According to this configuration, the gamma ray shield portion arranged between the neutron shield portion and the gamma ray detector in the lateral direction can prevent background gamma rays (e.g., gamma rays emitted from hydrogen elements in the neutron shield portion) from the neutron shield portion side from being incident on the gamma ray detector.

### Supplementary Note 9

The non-destructive inspection device according to Supplementary Note 8, wherein
the neutron generation portion, the neutron shield portion, the gamma ray shield portion, and the gamma ray detector are coupled directly or indirectly to each other.

### Advantageous Effect of Supplementary Note 9

With this configuration, the neutron generation portion, the neutron shield portion, the gamma ray shield portion, and the gamma ray detector can be integrally moved. Thus, the non-destructive inspection device can be easily moved and arranged relative to the inspection object.

### Supplementary Note 10

The non-destructive inspection device according to Supplementary Note 8 or 9, wherein
the gamma ray shield portion includes an inclined surface that faces in a direction inclined from the forward direction toward the gamma ray detector,
the inclined surface extends in such a way as to approach the gamma ray detector as a position shifts from a forward-direction front end of the gamma ray shield portion to a side opposite to the forward direction, and
each position on the inclined surface is located on a reference plane or located on a front side of the reference plane, the reference plane being an imaginary plane that contacts with both a front portion of the gamma ray detector and a front portion of the deceleration portion.

### Advantageous Effect of Supplementary Note 10

With this configuration, a size and a weight of the gamma ray shield portion can be reduced while the gamma ray shield portion can sufficiently exhibit a gamma ray shielding function.

Further, a straight advancement path of the gamma rays to the gamma ray detector from a predetermined location in the inspection object on a front side of the neutron generation portion is ensured to suppress interference between these gamma rays and the gamma ray shield portion. Thus, the gamma rays generated at the predetermined location and advancing to the gamma ray detector pass through the vicinity of the inclined surface along the inclined surface and enter the gamma ray detector. Accordingly, a large number of gamma rays from the predetermined location can be detected (e.g., selectively) by the gamma ray detector.

### Supplementary Note 11

The non-destructive inspection device according to any one of Supplementary Notes 8 to 10, further including:
a first neutron absorption portion that is arranged between the neutron shield portion and the gamma ray shield portion in the lateral direction and that is formed of a material absorbing neutrons.

### Advantageous Effect of Supplementary Note 11

With this configuration, neutrons are further prevented from entering a side of the gamma ray detector. Thereby, background gamma rays caused by neutrons entering a side of the gamma ray detector can be suppressed from being generated.

### Supplementary Note 12

The non-destructive inspection device according to any one of Supplementary Notes 8 to 11, further including:
a second neutron absorption portion including: a front absorption portion that is formed of a material absorbing neutrons and that extends in the lateral direction in such a way as to cover the gamma ray detector from a front side; and a lateral absorption portion that is formed of a material absorbing neutrons and that extend in the forward direction in such a way as to cover the gamma ray detector from a side of the neutron shield portion in the lateral direction, wherein
the lateral absorption portion is arranged between the gamma ray detector and the gamma ray shield portion in the lateral direction.

### Advantageous Effect of Supplementary Note 12

With this configuration, background gamma rays caused by neutrons entering a side of the gamma ray detector can be suppressed from being generated.

### Supplementary Note 13

The non-destructive inspection device according to any one of Supplementary Notes 8 to 12, further including:
a position adjustment mechanism for adjusting a position of the neutron generation portion, the neutron shield portion, the gamma ray shield portion, and the gamma ray detector; and
a base configured to support the neutron generation portion, the neutron shield portion, the gamma ray shield portion, and the gamma ray detector via the position adjustment mechanism,
wherein the position adjustment mechanism enables a position of the neutron generation portion, the neutron shield portion, the gamma ray shield portion, and the gamma ray detector to be adjusted relative to the base in a direction parallel to the forward direction.

### Advantageous Effect of Supplementary Note 13

According to this configuration, when a certain distance exists between the device (e.g., the neutron emission surface 13a1) and the surface of the inspection object, a distance from the inspection object to the neutron generation portion, the neutron shield portion, the gamma ray shield portion, and the gamma ray detector can be adjusted in a direction parallel to the forward direction. Thereby, the device can be positioned close to the surface of the inspection object, or detection data concerning a detection amount of the gamma rays derived from the target component in the inspection object can be optimized depending on a situation (e.g., a detection amount of the gamma rays from the vicinity of the surface of the inspection object can be increased by positioning the device to be slightly separated from a position of closely contacting with the surface of the inspection object).

### Supplementary Note 14

The non-destructive inspection device according to any one of Supplementary Notes 8 to 13, further including:
a position adjustment mechanism that enables a position of one of first and second structures to be adjusted relative to another of the first and second structures in a direction parallel to the forward direction, the first structure including the neutron generation portion, the neutron shield portion, and the gamma ray shield portion, and the second structure including the gamma ray detector.

### Advantageous Effect of Supplementary Note 14

According of this configuration, a position of one of the first structure including the neutron generation portion and the like and the second structure including the gamma ray detector can be adjusted relative to another of the first and second structures in the direction parallel to the forward direction. Thereby, detection data concerning a detection amount of the gamma rays derived from the target component in the inspection object can be optimized depending on a situation. For example, background gamma rays from a side of the neutron shield portion can be reduced so that an S/N ratio can be expected to be improved. Further, for example, calibration data (a calibration curve) and actual inspection data can be easily acquired in a state where first structure including the neutron generation portion and the like and the second structure including the gamma ray detector are in a predetermined positional relation in the direction parallel to the forward direction. Thus, the calibration data and the actual inspection data can be easily compared with each other.

### Supplementary Note 15

The non-destructive inspection device according to any one of Supplementary Notes 8 to 14, further including:
an inclination adjustment mechanism that enables an inclination of the neutron generation portion, the neutron shield portion, the gamma ray shield portion, and the gamma ray detector to be adjusted to a front side or a rear side.

### Advantageous Effect of Supplementary Note 15

According to this configuration, when a certain inclination exists between the device (e.g., the neutron emission surface 13a1) and the surface of the inspection object, an inclination of the neutron generation portion, the neutron shield portion, and the like relative to the inspection object can be adjusted by adjusting an angle of the neutron generation portion, the neutron shield portion, and the like with respect to the inspection object. Thereby, both of the device and the surface of the inspection object can be made parallel to each other, or detection data concerning a detection amount of the gamma rays derived from the target component in the inspection object can be optimized depending on a situation. For example, the inclination can be made to exist so that a detection amount of the gamma rays derived from the target component in an area somewhat deeper from the surface in the inspection object can be expected to be increased. Further, for example, calibration data (a calibration curve) and actual inspection data can be easily acquired in a state where the inclination is set at a predetermined inclination. Thus, the calibration data and the actual inspection data can be easily compared with each other.

### Supplementary Note 16

The non-destructive inspection device according to any one of Supplementary Notes 8 to 15, further including:
a distance adjustment mechanism that enables a distance from the gamma ray detector to the neutron shield portion and the gamma ray shield portion to be adjusted in the lateral direction.

### Advantageous Effect of Supplementary Note 16

According to this configuration, a lateral-direction distance from the gamma ray detector to the neutron shield portion and the gamma ray shield portion can be adjusted. Thereby, detection data concerning a detection amount of the gamma rays derived from the target component in the inspection object can be optimized depending on a situation.

### Supplementary Note 17

The non-destructive inspection device according to any one of Supplementary Notes 1 to 15, wherein
the gamma ray detector outputs the detection signal indicating energy of a gamma ray when the gamma ray enters the gamma ray detector,
the non-destructive inspection device further includes:
a detection amount measurement device configured to generate detection data indicating a detection amount of gamma rays for each energy of the gamma rays, based on each of the detection signals from the gamma ray detector; and
a sub-detector configured to cover the gamma ray detector from an area that is around the gamma ray detector, but that excludes at least an entry area of gamma rays from the inspection object, wherein when viewed from a front end portion of the forward direction in the gamma ray detector, the entry area is located on an obliquely front side that is a side inclined from the forward direction toward the neutron shield portion,
the sub-detector outputs an incidence signal when a gamma ray enters the sub-detector, and
the detection amount measurement device can be set in such a way as not to use the detection signal in generating the detection data when the detection signal and the incidence signal are output simultaneously.

### Advantageous Effect of Supplementary Note 17

With this configuration, the detection of gamma rays that are among gamma rays incident on the gamma ray detector and that are Compton-scattered and thereby incident on the sub-detector outside the gamma ray detector is not used in generating the detection data. Accordingly, the detection data with a less background can be generated.

When the sub-detector is formed of a material that shields gamma rays, the sub-detector also has a function of suppressing background gamma rays from entering from the gamma ray detector from an outside.

### REFERENCE SIGNS LIST

1: inspection object
1a: surface
10: non-destructive inspection device
11: neutron source (neutron generation portion)
12: gamma ray detector
13: neutron shield portion
13a: deceleration portion
13a1: neutron emission surface (front surface)
13a2: recess
13b: reflection portion
13b1: front surface
14: gamma ray shield portion
14a: front end surface (inclined surface)
14b: rear end surface
15: first neutron absorption portion
15a: front surface
16: second neutron absorption portion
16a: front absorption portion
16a1: front surface
16b: lateral absorption portion
17: liquid nitrogen dewar
18: cold finger
21: detection amount measurement device
22: preamplifier
23: data processing device
24: sub-detector
25: distance adjustment mechanism
25a: guide mechanism
25b: movable portion
31: support
31a: first support
31b: second support
31c: third support
31p: penetration hole
32: base
32a: first base
32b: second base
32b1: screw hole
32c: third base
33: position adjustment mechanism
33a: first position adjustment mechanism
33b: second position adjustment mechanism
34: bed
36: inclination adjustment mechanism
37: swing body
37a: penetration hole
37b: screw hole
38: first adjustment unit
38a: bolt
38b: first nut
38c: second nut
39: second adjustment unit
39a: bolt
39b: first nut
39c: second nut
41: first support member
42: second support member
51: connection unit
51a: attachment member
51b: bolt
51c: first nut
51d: second nut
61: spherical-surface portion
62: adjustment unit
62a: bolt
62b: nut
63: recess
64: spring
71: neutron generation tube
71a: ion source
71b: accelerator
71c: target (neutron generation portion)
71d: receptacle
72: power supply device
C1: first axis
C2: second axis
R: gamma ray entry area

What is claimed is:

1. A non-destructive inspection device comprising:
a neutron generation portion configured to spontaneously generate and emit neutrons or to generate and emit neutrons by DD nuclear fusion reaction or DT nuclear fusion reaction;
a neutron shield portion configured to cover the neutron generation portion from at least an area around the neutron generation portion and thereby shield the neutrons at the area around the neutron generation portion, and allow the neutrons to be emitted to a front side of the neutron generation portion; and
a gamma ray detector configured to detect gamma rays generated in an inspection object on a front side of the neutron generation portion, and output a detection signal concerning the detection, the gamma rays being generated as a result of the neutrons incident on the inspection object, wherein
the neutron shield portion and the gamma ray detector are arranged in alignment with each other in a lateral direction in relation to a forward direction, the forward direction being a direction from a rear side of the neutron generation portion to a front side of the neutron generation portion.

2. The non-destructive inspection device according to claim 1, wherein
the neutron generation portion is a neutron source configured to spontaneously generate and emit neutrons, and
the neutron shield portion is configured to cover the neutron source from the area around and a rear side of the neutron source and shield the neutrons at the area around and the rear side of the neutron source, and allow the neutrons to be emitted to the front side of the neutron source.

3. The non-destructive inspection device according to claim 1, further comprising:
a neutron generation tube configured to generate the neutrons by the DD nuclear fusion reaction or the DT nuclear fusion reaction, wherein
the neutron generation portion is a target that is arranged inside a front end portion of the neutron generation tube and that generates the neutrons by the DD nuclear fusion reaction or the DT nuclear fusion reaction caused by deuterium ions or tritium ions colliding with the target, and
the neutron shield portion covers the front end portion of the neutron generation tube and the target from the area around the front end portion.

4. The non-destructive inspection device according to claim 1, wherein
the neutron shield portion includes
a deceleration portion that is formed of a material decelerating the neutrons and that covers the neutron generation portion from at least the area around the neutron generation portion, and
a reflection portion that is formed of a material reflecting the neutrons and that covers the deceleration portion from an area around and a rear side of the deceleration portion.

5. The non-destructive inspection device according to claim 2, wherein
the neutron shield portion includes
a deceleration portion that is formed of a material decelerating the neutrons and that covers the neutron source from the area around and a rear side of the neutron source, and
a reflection portion that is formed of a material reflecting the neutrons and that covers the deceleration portion from an area around and a rear side of the deceleration portion, and
the neutron source is arranged at a front end portion of the deceleration portion in the forward direction.

6. The non-destructive inspection device according to claim 2, wherein
the neutron shield portion includes
a deceleration portion that is formed of a material decelerating the neutrons and that covers the neutron source from the area around and a rear side of the neutron source, and
a reflection portion that is formed of a material reflecting the neutrons and that covers the deceleration portion from an area around and a rear side of the deceleration portion, and
the neutron source is arranged at a position shifted toward the gamma ray detector from a lateral-direction center of the deceleration portion when viewed in a direction opposite to the forward direction.

7. The non-destructive inspection device according to claim 4, wherein
the deceleration portion includes a neutron emission surface that faces in the forward direction and that emits the neutrons from the neutron generation portion.

8. The non-destructive inspection device according to claim 4, further comprising:
a gamma ray shield portion that is arranged between the neutron shield portion and the gamma ray detector in the lateral direction and that shields gamma rays.

9. The non-destructive inspection device according to claim 8, wherein
the neutron generation portion, the neutron shield portion, the gamma ray shield portion, and the gamma ray detector are coupled directly or indirectly to each other.

10. The non-destructive inspection device according to claim 8, wherein
the gamma ray shield portion includes an inclined surface that faces in a direction inclined from the forward direction toward the gamma ray detector,
the inclined surface extends in such a way as to approach the gamma ray detector as a position shifts from a forward-direction front end of the gamma ray shield portion to a side opposite to the forward direction, and
each position on the inclined surface is located on a reference plane or located on a front side of the reference plane, the reference plane being an imaginary plane that contacts with both a front portion of the gamma ray detector and a front portion of the deceleration portion.

11. The non-destructive inspection device according to claim 8, further comprising:
a first neutron absorption portion that is arranged between the neutron shield portion and the gamma ray shield portion in the lateral direction and that is formed of a material absorbing neutrons.

12. The non-destructive inspection device according to claim 8, further comprising:
a second neutron absorption portion including: a front absorption portion that is formed of a material absorbing neutrons and that extends in the lateral direction in such a way as to cover the gamma ray detector from a front side; and a lateral absorption portion that is formed of a material absorbing neutrons and that extend in the forward direction in such a way as to cover the gamma ray detector from a side of the neutron shield portion in the lateral direction, wherein
the lateral absorption portion is arranged between the gamma ray detector and the gamma ray shield portion in the lateral direction.

13. The non-destructive inspection device according to claim 8, further comprising:
a position adjustment mechanism for adjusting a position of the neutron generation portion, the neutron shield portion, the gamma ray shield portion, and the gamma ray detector; and
a base configured to support the neutron generation portion, the neutron shield portion, the gamma ray shield portion, and the gamma ray detector via the position adjustment mechanism,
wherein the position adjustment mechanism enables a position of the neutron generation portion, the neutron shield portion, the gamma ray shield portion, and the gamma ray detector to be adjusted relative to the base in a direction parallel to the forward direction.

14. The non-destructive inspection device according to claim 8, further comprising:
a position adjustment mechanism that enables a position of one of first and second structures to be adjusted relative to another of the first and second structures in a direction parallel to the forward direction, the first structure including the neutron generation portion, the neutron shield portion, and the gamma ray shield portion, and the second structure including the gamma ray detector.

15. The non-destructive inspection device according to claim 8, further comprising:
an inclination adjustment mechanism that enables an inclination of the neutron generation portion, the neutron shield portion, the gamma ray shield portion, and the gamma ray detector to be adjusted to a front side or a rear side.

16. The non-destructive inspection device according to claim 8, further comprising:
a distance adjustment mechanism that enables a distance from the gamma ray detector to the neutron shield portion and the gamma ray shield portion to be adjusted in the lateral direction.

17. The non-destructive inspection device according to claim 1, wherein
the gamma ray detector outputs the detection signal indicating energy of a gamma ray when the gamma ray enters the gamma ray detector,
the non-destructive inspection device further comprises:
a detection amount measurement device configured to generate detection data indicating a detection amount of gamma rays for each energy of the gamma rays, based on each of the detection signals from the gamma ray detector; and
a sub-detector configured to cover the gamma ray detector from an area that is around the gamma ray detector, but that excludes at least an entry area of gamma rays from the inspection object, wherein when viewed from a front end portion of the forward direction in the gamma ray detector, the entry area is located on an obliquely front side that is a side inclined from the forward direction toward the neutron shield portion,
the sub-detector outputs an incidence signal when a gamma ray enters the sub-detector, and
the detection amount measurement device can be set in such a way as not to use the detection signal in generating the detection data when the detection signal and the incidence signal are output simultaneously.
